(19) United States
(12) Patent Application Publication
(10) Pub. No.: US 2011/0014328 A1
(43) Pub. Date: Jan. 20, 2011

(54) FUNCTIONALLY SUPERIOR WHEY PROTEINS

(75) Inventors: Syed S. H. RIZVI, Ithaca, NY (US); Khanitta MANOI, Nakornsanway (TH)

Correspondence Address: HESLIN ROTHENBERG FARLEY & MESITI PC, 5 COLUMBIA CIRCLE, ALBANY, NY, 12203, US

(73) Assignee: CORNELL UNIVERSITY, Ithaca, NY (US)

(21) Appl. No.: 12/824,277

(22) Filed: Jun. 28, 2010

Related U.S. Application Data
(60) Provisional application No. 61/220,999, filed on Jun. 26, 2009.

(51) Int. Cl. A23J 3/08; A23J 1/20; A23D 7/02; A23L 1/24; G01N 33/02

(52) U.S. Cl. 426/231; 426/657; 426/648; 426/602; 426/605; 426/603; 426/570; 426/474

(57) ABSTRACT

The present invention relates to a texturized whey protein composition having enhanced functional properties, including, for example, enhanced viscosifying properties, enhanced emulsifying properties, and/or enhanced gelling properties. In one embodiment, the texturized whey protein composition is a supercritical fluid extrusion product derived from whey protein. The present invention also relates to a process for preparing the texturized whey protein composition, where the process involves a supercritical fluid extrusion process. The present invention further relates to various products containing the texturized whey protein composition.

The present application claims benefit of U.S. Provisional Patent Application Ser. No. 61/220,999, filed Jun. 26, 2009, which is hereby incorporated by reference in its entirety.

### FIELD OF THE INVENTION

The present invention relates to a texturized whey protein composition having enhanced functional properties. The present invention also relates to a process for preparing the texturized whey protein composition. The present invention further relates to various products containing the texturized whey protein composition.

### BACKGROUND OF THE INVENTION

The development of new approaches to better utilize food ingredients in general and dairy ingredients in particular is a major challenge in today's competitive world. Whey proteins (WPs) are widely used in a variety of food formulations and constitute a significant share of the dairy ingredients market. Their ability to gel upon heating provides desirable food textures which are important to consumer acceptability.

Although numerous studies on WP modification have been reported, several barriers still remain. For example, heat-induced gelation of WPs has been extensively studied and used to add texture to food products (Mangino, 1992; Mulvihill & Kinsella, 1987; Paulsson, Hegg, & Castberg, 1986; Ziegler & Foegeding, 1990). However, foods containing WPs had to be heated above 65° C. before the proteins would form gels or thicken solutions. This limited their application in many types of products containing heat sensitive-ingredients.

Whey proteins have also been reported to aid in the formation and stabilization of oil-in-water (o/w) emulsions (Mangino, 1984; Schmidt, Packard, & Morris, 1983) and this functionality is related to their interfacial area and adsorption at the water-oil interface (Dalgleish, 1997; Dickinson, 1997; Pearce & Kinsella, 1978). During emulsification, WP adsorption at the water-oil interface is further improved due to hydrophobic interactions between a section of the proteins and the oil surface. This creates an interfacial layer which is generally charged (because proteins contain charged amino acids) and can also sterically stabilize the oil droplets (Dalgleish, 1997).

However, emulsion gels produced by heat treatment have limited uses, especially for food formulations containing heat-sensitive ingredients. The appearance of emulsion gels prepared by salt-induced gelation is highly dependent on calcium concentrations. Increasing salt concentration produced a particulate gel with poor water holding capacity (Boutin et al., 2007). Additionally, preparation of emulsion gels by salt or acid-induced gelation process is a time consuming process and it is difficult to control the final gel texture.

There is considerable interest in converting oil-in-water (o/w) emulsions stabilized by WPs into gels which can be used to create foods with improved organoleptic properties. These gels are often produced by heat treatment of WP-stabilized emulsions. However, as noted above, such heat treatment processes limit the use of WP gels in food formulations containing heat-sensitive ingredients.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic of one embodiment of a process of the present invention and apparatus for whey protein concentrate (WPC)-based texturization by supercritical fluid extrusion (SCFX), screw configuration, and temperature zones. In a particular embodiment, texturization of WPC80 by supercritical fluid extrusion can be performed. A suitable extruder can include a Wenger TX-52 Magnum co-rotating twin-screw extruder.

FIGS. 2A-2B are graphs showing variation of apparent viscosity with shear rate for dispersions containing 20% (w/w) tWPC powders extruded at different pH; (a) without SC—CO₂ (FIG. 2A); and (b) with 1% (db, dry basis) SC—CO₂ injection (FIG. 2B) and unprocessed WPC blend powder. Data are averages for 3 tests. The maximum error in apparent viscosity data was 5%. *tWPC powder extruded with water only (non-pH-adjusted).

FIGS. 3A-3B are graphs showing shear stress-shear rate relationship of dispersions containing 20% (w/w) tWPC powders extruded at different pH; (a) without SC—CO₂ (FIG. 3A) and (b) with 1% (db) SC—CO₂ injection (FIG. 3B) and unprocessed WPC blend powder. Data are averages for 3 tests. The maximum error in shear stress-shear rate data was 5%. *tWPC powder extruded with water only (non-pH-adjusted).

FIGS. 4A-4B are graphs showing variation of apparent viscosity (at 61.15 s⁻¹ shear rate) with WP concentrations for dispersions containing tWPC powders extruded at different pH; (a) without SC—CO₂ (FIG. 4A), and (b) with 1% (db) SC—CO₂ injection (FIG. 4B) and unprocessed WPC blend powder. Error bars represent standard deviation from the mean of 3 trials. *tWPC powder extruded with water only (non-pH-adjusted).

FIGS. 5A-5B are graphs showing variation of storage (elastic) modulus (G′) with frequency for dispersions containing 30% (w/w) tWPC powders extruded at different pH; (a) without SC—CO₂ (FIG. 5A), and (b) with 1% (db) SC—CO₂ injection (FIG. 5B). Data are averages for 3 tests. The maximum error in G′ data was 5%. *tWPC powder extruded with water only (non-pH-adjusted).

FIGS. 6A-6B are graphs showing variation of tan delta (tan δ) with frequency for dispersions containing 30% (w/w) tWPC powders extruded at different pH; (a) without SC—CO₂ (FIG. 6A), and (b) with 1% (db) SC—CO₂ injection (FIG. 6B). Data are averages for 3 tests. The maximum error in tan δ data was 5%. *tWPC powder extruded with water only (non-pH-adjusted).

FIG. 7 is a graph showing temperature sweep test results of selected WP dispersions (20%, w/w). G′ and G″ (▴,Δ) for dispersion containing tWPC powder extruded at pH 2.89 with 1% (db) SC—CO₂ injection; G′ and G″ (▪,□) for dispersion containing tWPC powder extruded at pH 8.16 with 1% (db) SC—CO₂ injection; G′ and G″ (,∘) for WP dispersion containing unprocessed WPC blend powder. Data are averages for 3 tests. The maximum error in G′ and G″ data was 5%.

FIG. 8 is a graph showing water holding capacity (WHC) of selected tWPC powders at 25° C. Error bars represent standard deviation from the mean of 3 trials.

FIG. 9 is a graph showing % Creaming index of emulsions (20%, w/w oil) stabilized by commercial WPC80 and tWPC at different concentrations in aqueous phase and after storage for 1, 7, and 14 days at ambient temperature. Data are reported as an average of three replications with the maximum error of 5%.

FIG. 10 is a photograph showing emulsions stabilized by commercial WPC80 (top), and tWPC (bottom) at various oil mass fractions (φ).

FIGS. 11A-11B are graphs showing variation of apparent viscosity with shear rate at 25° C. for emulsions stabilized by (a) commercial WPC80 (FIG. 11A), and (b) tWPC (FIG. 11B) at various oil mass fractions (φ=0.20 to 0.80). The maximum error in data was 5%. (The symbol φ may also be referenced in the figures using the letter “f.”)

FIG. 12 is a graph showing variations of elastic (G′, solid lines) and loss (G″, dash lines) moduli with frequency at 25° C. for emulsions stabilized by tWPC at different oil mass fractions (φ=0.20 to 0.80). The maximum error in data was 5%.

FIG. 13 is a graph showing variation of loss tangent (tan δ) with frequency at 25° C. for emulsions stabilized by tWPC at different oil mass fractions (φ=0.20 to 0.80). The maximum error in data was 5%.

FIGS. 14A-14C are confocal laser scanning microscopy (CLSM) images of emulsions stabilized by tWPC at oil mass fraction of (a) 0.80 (FIG. 14A); (b) 0.50 (FIG. 14B); and (c) 0.2 (FIG. 14C). Emulsions were stained with a mixture of Nile red (indicated fat phase, green color) and Fast Green FCF (indicated protein phase, red color). The bar represents 13.5 μm.

FIGS. 15A-15C are graphs showing Cox-Merz correlation between oscillatory and steady shear responses for emulsions stabilized by tWPC at selected oil mass fractions of 0.80 (FIG. 15A), 0.50 (FIG. 15B), and 0.20 (FIG. 15C) at 25° C.

FIG. 16 is a graph showing variations of elastic modulus (G′) with temperature for emulsions stabilized by tWPC at different oil mass fractions (φ=0.20 to 0.80). The maximum error in data was 5%.

### SUMMARY OF THE INVENTION

According to one aspect, the present invention provides a texturized whey protein composition (tWPC). In one embodiment, the texturized whey protein composition is a supercritical fluid extrusion product derived from whey protein.

According to another aspect, the present invention provides a food product, where the food product includes a texturized whey protein composition of the present invention.

According to another aspect, the present invention provides a food preparation agent, where the food preparation agent includes a texturized whey protein composition of the present invention. Suitable food preparation agents can include, without limitation, a thickening agent, a gelling agent, a stabilizer, a water binder, and the like.

According to another aspect, the present invention provides an emulsifier for use in food preparations, where the emulsifier includes a texturized whey protein composition of the present invention.

According to another aspect, the present invention provides an oil-in-water emulsion. In one embodiment, the oil-in-water emulsion includes (i) an aqueous phase containing a food emulsifier that includes a texturized whey protein composition of the present invention; and (ii) an oil phase dispersed in the aqueous phase.

According to another aspect, the present invention provides a food product that includes an oil-in-water emulsion of the present invention.

According to another aspect, the present invention provides a method of preparing an edible cold-setting gel-like emulsion. This method involves (i) providing an aqueous phase containing a texturized whey protein composition of the present invention; and (ii) dispersing an oil phase in the aqueous phase under conditions effective to yield an edible cold-setting gel-like emulsion.

According to another aspect, the present invention provides a process for preparing a texturized whey protein composition. This process involves (i) providing a whey protein mixture that includes a whey protein concentrate; and (ii) subjecting the whey protein mixture to a supercritical fluid extrusion (SCFX) process under conditions effective to yield a texturized whey protein composition having enhanced functional properties compared to a non-texturized whey protein composition.

According to another aspect, the present invention provides a texturized whey protein composition prepared by the process of the present invention.

The texturized whey protein composition of the present invention is useful for a variety of food applications. Due to its high nutritional value and functional properties, the tWPC of the present invention can be used in various food products and in various methods of preparing food products. For example, the tWPC of the present invention can be used in infant formulas, food supplements, sport bars, sports beverages, and the like. Whey protein now constitutes a share of the dairy ingredient market, and, therefore, the tWPC of the present invention can be used in this market.

Whey protein is of interest due to its ability to gel upon heating (though cold gelling whey is also of high interest); this gelling provides food textures that are important to consumers. While whey proteins naturally possess some emulsifying properties, the surface-active functionality performance and stability of whey proteins are highly dependent on many factors such as surface hydrophobicity, protein flexibility, heat treatment, pH of the medium, ionic strength, solubility, and protein concentration. Therefore, such characteristics of whey proteins have limited the use of whey proteins in many products. In contrast, the tWPC of the present invention and the process for preparing the tWPC overcome such deficiencies of other whey proteins and whey protein preparations in the art.

As provided herein, the present invention relates to processes, preparations, and uses of a functional whey protein preparation for various food applications. In a particular embodiment, this process involves supercritical fluid extrusion of a texturized whey protein composition with CO₂. Unlike most other whey protein preparations, the present invention provides a whey protein preparation that is cold-gelling, has thermal stability (unlike most other whey preparations), high solubility, high viscosity, and a creamy texture. The texturized whey protein composition of the present invention can enhance the emulsifying activity and stability in oil-in-water emulsions, allowing the texturized whey protein composition to be used as a thickening/gelling agent, fat substitute or emulsifier. The texturized whey protein preparation of the present invention can be used in applications where heating is not desired, such as, but not limited to mayonnaise, the production of spreadable butter without the need to add oil, and in baked goods and other foods where heating is required.

The texturized whey protein composition was observed to possess extremely high viscosity with the ability to form a viscoelastic gel at ambient temperature. The unique functionalities of this texturized whey protein composition, such as high solubility, thickening properties, superior water holding capacity, and high surface hydrophobicity, have indicated the possibility of utilizing this ingredient as a polymeric surfactant or emulsifier for food emulsions.

The present invention is unique, in that it provides, for the first time, cold, gel-like emulsions prepared with texturized whey protein concentrate (e.g., WPC80) at ambient temperature. Therefore, the texturized whey protein composition of the present invention is useful for controlling the texture of emulsion-filled gel products and their derivatives.

### DETAILED DESCRIPTION OF THE INVENTION

Whey proteins (WPs) are used in a variety of food formulations and constitute a significant share of the dairy ingredients market.

The present invention is based, in part, on the discovery that supercritical fluid extrusion (SCFX) of whey protein is effective to yield a texturized whey protein composition that has superior functionality compared to conventional whey protein compositions, including conventional whey protein concentrates that have or have not undergone some sort of process to enhance functionality (e.g., non-supercritical fluid extrusion processes or supercritical fluid extrusion processes). Therefore, as described in more detail herein, the present invention relates to a texturized whey protein composition having superior functionality, various food products and food preparation agents containing the texturized whey protein composition, methods of making such food products, and processes for using supercritical fluid extrusion technology to yield a texturized whey protein composition having superior functionality.

In one aspect, the present invention relates to a texturized whey protein composition (referred to herein as “tWPC”).

As used herein, unless otherwise defined, the term “texturized” refers to a modified composition, product, concentrate, and/or naturally occurring composition (e.g., a texturized whey protein composition made from whey protein, whey protein concentrate, whey protein isolate, and the like) that has been modified to give it a desired texture. In one embodiment, the modification can be achieved by a particular process or treatment (e.g., physical, chemical, and/or enzymetical methods, or a combination of such methods). The term “texturized food” can also refer to a manufactured food product designed to imitate another food, e.g., in order to give the manufactured food product a characteristic that is equal to or superior to the food being imitated (e.g., a texturized vegetable protein that is spun or extruded to simulate meat).

In a particular embodiment, the texturized whey protein composition of the present invention is a supercritical fluid extrusion product derived from whey protein. Therefore, the present invention contemplates any texturized whey protein composition that has undergone supercritical fluid extrusion processing.

Examples of suitable whey protein sources can include, without limitation, any whey protein in the form of a whey protein concentrate, a whey protein isolate, or a whey protein hydrolysate, and any mixture containing whey protein. Whey proteins of these various forms are well known in the art and are commercially available.

In one embodiment, the supercritical fluid extrusion product can include about 70 or more weight percent of a whey protein concentrate. In a particular embodiment, the supercritical fluid extrusion product can include whey protein concentrate in an amount of between about 70 and about 100 weight percent; between about 72 and about 98 weight percent; between about 74 and about 96 weight percent; between about 76 and about 94 weight percent; between about 78 and about 92 weight percent; between about 80 and about 90 weight percent; between about 82 and about 88 weight percent; and between about 84 and about 86 weight percent. Therefore, the present invention contemplates supercritical fluid extrusion products having any weight percent of a whey protein concentrate of between about 70 and about 100 weight percent (e.g., 70, 71, 72, 73, etc.).

A particular whey protein concentrate suitable for the present invention can include, for example, whey protein concentrate 80 (referred to as “WPC80”) (Davisco Foods International, Inc., Le Sueur, Minn.). However, other suitable whey protein sources can be used to prepare the tWPC of the present invention, including other whey protein concentrates, whey protein isolates, and the like.

In another embodiment, the supercritical fluid extrusion product can include a whey protein concentrate and an edible polysaccharide component. The whey protein concentrate and edible polysaccharide component can be present at various percent by weight concentrations, where the whey protein concentrate is present in an amount of about 70 or more weight percent, and where the edible polysaccharide component is present in an amount of about 15 or less weight percent.

For example, the whey protein concentrate can be present in an amount as described herein above, while the edible polysaccharide component can be present in an amount of between about 0 and about 15.0 weight percent; between about 0.5 and about 14.5 weight percent; between about 1.0 and about 14.0 weight percent; between about 1.5 and about 13.5 weight percent; between about 2.0 and about 13.0 weight percent; between about 2.5 and about 12.5 weight percent; between about 3.0 and about 12.0 weight percent; between about 3.5 and about 11.5 weight percent; between about 4.0 and about 11.0 weight percent; between about 4.5 and about 10.5 weight percent; between about 5.0 and about 10.0 weight percent; between about 5.5 and about 9.5 weight percent; between about 6.0 and about 9.0 weight percent; between about 6.5 and about 8.5 weight percent; and between about 7.0 and about 8.0 weight percent. Thus, the present invention contemplates whey protein concentrate to be present in an amount of between about 70 and about 100 weight percent (as described herein above), and edible polysaccharide component to be present in an amount of between about 0 and about 15 weight percent. Thus, the present invention contemplates embodiments in which the edible polysaccharide component is present in any amount between about 0 and about 15 weight percent, including 0, 1, 2, 3, 4, 5, 6, 7, 8, 9, 10, 11, 12, 13, 14, and 15 weight percent, and degrees thereof as measured in 10ths of a weight percent (e.g., 1.2 weight percent) and/or 100ths of a weight percent (e.g., 15.25 weight percent).

As used herein, a suitable edible polysaccharide component can be any polysaccharide that is edible by humans or animals. A particular edible polysaccharide component can include, for example, a starch. Examples of suitable starches can include, without limitation, corn, potato, rice, tapioca, bran, and soy starches, modified variants thereof, and mixtures thereof.

The texturized whey protein composition of the present invention has enhanced functional properties.

As used herein, the term “enhanced functional properties” refers to enhanced viscosifying properties, enhanced emulsifying properties, and/or enhanced gelling properties of the texturized whey protein composition of the present invention (or products containing the tWPC) as compared to whey proteins that have not undergone supercritical fluid extrusion, whether or not the non-supercritcal fluid extruded whey proteins are in the form of whey protein concentrates, whey protein isolates, or whey protein hydrolysates.

Various tests well known in the art can be used to determine whether the texturized whey protein composition of the present invention (or products containing the tWPC) has enhanced functional properties, as described in more detail below.

As used herein, the term “ambient temperature” refers to room temperature (e.g., between about 20° C. to about 25° C.) or a temperature in which no external heat energy has been applied to the subject process, method, composition, and/or product of the present invention, including any tests to determine viscosifying, emulsifying, and/or gelling properties.

As used herein, the term “enhanced viscosifying properties” refers to the ability of an agent to give “thickness” or “viscosity” to a product (e.g., a food product, an oil-in-water emulsion, or the like), or to provide resistance of the product to pouring. Viscosifying agents are used in foods as thickeners to produce improved mouthfeel and as stabilizers to prevent settling out of particulate matter.

In a particular embodiment, the texturized whey protein composition of the present invention has enhanced viscosifying properties as exhibited by a reference sample of between about 10 and about 20 weight percent of the texturized whey protein composition having an apparent viscosity of between about 0.07 and about 2.06 pascal-seconds (Pa·s) measured at a shear rate of about 25 s⁻¹ and at ambient temperature.

As used herein, the term “enhanced emulsifying properties” refers to the ability of an agent to stabilize a mixture of two or more immiscible liquids in which one is dispersed in the other as microscopic or ultramicroscopic droplets. Emulsions can be stabilized by agents (e.g., emulsifiers) that form films at the droplets' surface or impart mechanical stability. Less stable emulsions eventually separate spontaneously into two liquid layers; more stable ones can be destroyed by inactivating the emulsifier, by freezing, or by heating.

In one embodiment, the texturized whey protein composition of the present invention has enhanced emulsifying properties as compared to commercially available whey protein compositions (e.g., WPC80 and the like). In a particular embodiment, the texturized whey protein composition of the present invention has enhanced emulsifying properties as exhibited by a reference sample having about 20 weight-in-weight (w/w) percent oil concentration and about 3 percent weight-in-weight (w/w) of the texturized whey protein composition of the present invention in an aqueous phase, where the reference sample exhibits an emulsifying activity index (EAI) and/or an emulsion stability index (ESI) of greater quality than that of a commercial whey protein composition or a whey protein composition that has not been texturized using an SCFX process.

A suitable whey protein composition for use in comparison tests against the texturized whey protein composition of the present invention for enhanced emulsifing properties can include, for example, the whey protein known in the art as WPC80, which is commercially available. Commercial WPC80 was found to have an ESI of around 30 hours (e.g., about 33 hours). Thus, according to one embodiment, the texturized whey protein composition of the present invention has enhanced emulsifying properties (compared to a non-texturized whey protein composition) as exhibited by a reference sample thereof having an ESI of at least about 30 hours or greater.

In another embodiment, the texturized whey protein composition of the present invention has enhanced emulsifying properties (compared to a non-texturized whey protein composition) as exhibited by a reference sample thereof having an ESI of at least the following hours: 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, or 12000 hours or greater.

In a particular embodiment, the texturized whey protein composition of the present invention has enhanced emulsifying properties as exhibited by a reference sample having about 20 weight-in-weight (w/w) percent oil concentration and about 3 percent weight-in-weight (w/w) of the texturized whey protein composition of the present invention in an aqueous phase, where the reference sample exhibits an EAI of at least about 22 m²/g and an ESI of at least about 12,000 hours measured at ambient temperature. In a more particular embodiment, the ESI of the texturized whey protein composition is at least about 12,100 hours; at least about 12,200 hours; at least about 12,300 hours; at least about 12,400 hours; at least about 12,500 hours; at least about 12,600 hours; at least about 12,700 hours; at least about 12,800 hours; at least about 12,900 hours; at least about 13,000 hours; at least about 13,100 hours; at least about 13,200 hours; at least about 13,300 hours; at least about 13,400 hours; at least about 13,500 hours; and more particularly at least about 13,504 hours.

As used herein, the term “enhanced gelling properties” refers to the ability of a disperse phase to combine with a dispersion medium to produce a semi-solid or self-standing material.

The texturized whey protein composition of the present invention can be used in any food preparation application calling for whey protein. In one embodiment, the texturized whey protein composition of the present invention is effective as a food preparation agent, including, without limitation, food preparation agents such as thickening agents, gelling agents, emulsifiers, stabilizers, and water binders.

In another aspect, the present invention relates to a process for preparing a texturized whey protein composition, where the texturized whey protein composition has enhanced functional properties. This process involves (i) providing a whey protein mixture that includes a whey protein concentrate; and (ii) subjecting the whey protein mixture to a supercritical fluid extrusion process under conditions effective to yield a texturized whey protein composition having enhanced functional properties compared to a non-texturized whey protein composition. Various embodiments and aspects of this process are described herein.

SCFX is an innovative food processing technology that offers sub-100° C. expansion using direct supercritical fluid carbon dioxide (SC—CO₂) injection. Its distinct low-temperature and low-shear conditions due to high moisture allow for the retention of heat sensitive ingredients. The general expansion mechanism of an SCFX process consists of the following major steps: (a) development of gas holding matrix by heat-shear treatment, (b) injection of SC—CO₂ into the matrix and mixing in the extruder barrel to create a saturated solution, (c) nucleation of cells induced by thermodynamic instability created by a sudden pressure drop at the die, (d) cell growth and extrudate expansion at the die exit as the pressure quenches to atmospheric level (Alavi, Gogoi, Khan, Bowman, & Rizvi, 1999; Mulvaney & Rizvi, 1993). The delicate balance of temperature and shear during SCFX processing permit controlled modification of WP conformation. The SC—CO₂ is an environmentally friendly solvent and is chemically inert which is ideal for food processing. Addition of SC—CO₂ provides additional acidic environment and also serves as blowing agent for surface modification of WP matrices.

As set forth above, the enhanced functional properties of the texturized whey protein composition produced by the process of the present invention can include, without limitation, enhanced viscosifying properties, enhanced emulsifying properties, and/or enhanced gelling properties.

As used herein, providing a whey protein mixture that includes a whey protein concentrate can involve providing whey protein concentrate in the amounts described herein above. The present invention also contemplates using whey protein isolates or whey protein hydrolysates in place of or in addition to a whey protein concentrate to arrive at the desired concentration of whey protein in the whey protein mixture to be provided.

In one embodiment of the process for preparing the tWPC of the present invention, the whey protein mixture includes about 70 or more weight percent of the whey protein concentrate. Suitable variations of the concentration of whey protein concentrate are as described herein.

In another embodiment of the process for preparing the tWPC of the present invention, the whey protein mixture can further include an edible polysaccharide component (as described herein).

In a particular embodiment of the process for preparing the tWPC of the present invention, the edible polysaccharide component is present in an amount of about 15 or less weight percent. Suitable variations of the concentration of the edible polysaccharide component are as described herein.

In another embodiment of the process for preparing the tWPC of the present invention, the whey protein mixture can include between about 70 and about 100 weight percent of the whey protein concentrate and between about 0 and about 15 weight percent of the edible polysaccharide component. Suitable variations of the concentration of the whey protein concentrate and of the edible polysaccharide component are as described herein.

As used herein, subjecting the whey protein mixture to an SCFX process under conditions effective to yield a texturized whey protein composition having enhanced functional properties compared to a non-texturized whey protein composition can be carried out using an SCFX apparatus as described in U.S. Pat. No. 5,120,559 to Rizvi et al. and U.S. Pat. No. 5,417,992 to Rizvi et al., the disclosures of which are hereby incorporated by reference in their entirety. However, modifications to the SCFX process as described herein are suitable to yield a texturized whey protein composition of the present invention having the enhanced functional properties described herein.

In one embodiment of the process of preparing the tWPC of the present invention, the SCFX process includes introducing supercritical fluid carbon dioxide (SC—CO2) under conditions effective to aid in the production of the texturized whey protein composition.

In one embodiment, the SCFX process is carried out without the external addition of any heat energy. However, the present invention contemplates embodiments that include the addition of heat energy. In a particular embodiment, the SCFX process of the present invention can be carried out at a temperature of between about 15° C. and about 100° C., and more particularly between about 20° C. and about 95° C., between about 25° C. and about 90° C., between about 30° C. and about 85° C., between about 35° C. and about 80° C., between about 40° C. and about 75° C., between about 45° C. and about 70° C., between about 50° C. and about 65° C., and between about 55° C. and about 60° C. In a particular embodiment, the SCFX process is carried out at ambient temperature.

In one embodiment, the SCFX process is carried out at any pH, ranging from a highly acidic to a highly alkaline pH. In a particular embodiment, the SCFX process is carried out at a pH of between about 2.0 and about 8.4, and more particularly at a pH of about 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, and 8.4. In a particular embodiment, the SCFX process is carried out at a pH of between about 2.80 and about 3.00, and more particularly at a pH of about 2.89.

The process of preparing a tWPC of the present invention contemplates the use of an SCFX process that is carried out at any of the temperature and pH values described herein.

In one embodiment of the process of preparing the tWPC of the present invention, the SCFX process is carried out at a temperature of between about 20° C. and about 100° C. and at a pH of about 3.5 or lower.

Methods for adjusting the pH are well known by those of ordinary skill in the art. As used in the SCFX process of the present invention, adjusting the pH of the whey protein mixture to a value of about 3.5 or lower can be achieved by adding an acid, introducing carbon dioxide under high pressure, or a combination thereof to the whey protein mixture during the SCFX process (e.g., while the whey protein mixture is in the SCFX apparatus). Suitable acids for use in adjusting the pH used in the SCFX process to an acidic pH, and more particularly in adjusting the pH of the whey protein mixture to an acidic pH, can include, without limitation, hydrochloric acid, sulfuric acid, phosphoric acid, acetic acid, lactic acid, and the like, and combinations thereof. Adjusting the pH used in the SCFX process to a more basic pH, and more particularly in adjusting the pH of the whey protein mixture to a more basic pH, can be done using well known techniques in the art.

In another embodiment of the process of preparing the tWPC of the present invention, the SCFX process is carried out at a pH of about 3.5 or lower, at a temperature of between about 85° C. and about 95° C., and at a pressure of between about 10 and 15 megapascal (MPa).

In another embodiment of the process of preparing the tWPC of the present invention, the whey protein mixture can further include at least one inorganic element-containing compound. Suitable amounts of the at least one inorganic element-containing compound include amounts sufficient to prevent excessive aggregation and to promote a stronger structure of protein gels, as illustrated herein. Suitable inorganic elements can include, for example, calcium, sodium, potassium, magnesium, zinc, manganese, and the like. More particularly, the at least one inorganic element-containing compound can include, without limitation, calcium carbonate, calcium chloride, calcium gluconate, calcium lactate, sodium chloride, sodium citrate, sodium acetate, sodium lactate, and mixtures thereof.

In another embodiment of the process of preparing the tWPC of the present invention, the process can further involve drying the texturized whey protein composition. Suitable drying techniques are well known in the art.

In another embodiment of the process of preparing the tWPC of the present invention, the process can further involve grinding the texturized whey protein composition into a powder. Suitable grinding techniques are well known in the art.

In another aspect, the present invention relates to a texturized whey protein composition prepared by the process described herein.

In another aspect, the present invention relates to a food product, where the food product includes a texturized whey protein composition of the present invention. Examples of food products that can include the tWPC of the present invention include, but are not limited to, protein-enriched food products, nutrition bars (e.g., sports bars or other bars), dietary supplements, reduced fat formulated food products, and the like. One of ordinary skill in the art would readily understand how the tWPC can be used in preparing these food products. Therefore, in one embodiment, the present invention contemplates using the tWPC of the present invention as a protein substitute for any food product having protein of any form as an added ingredient, as evidenced, for example, by a listing of a protein in the list of ingredients on the label of a food product. Examples of proteins for which the tWPC can serve as a substitute include, without limitation, any form (e.g., a protein concentrate, a protein isolate) of plant protein, animal protein, and/or dairy protein (e.g., milk and cheese proteins), including, for example, whey protein, soy protein, milk protein, pea protein, rice protein, egg protein (e.g., egg yolk protein, egg white protein), potato protein, casein, sodium caseinate, calcium caseinate, lupin protein, wheat protein, corn protein, and the like.

In another aspect, the present invention relates to a food preparation agent, where the food preparation agent includes a texturized whey protein composition of the present invention. Suitable food preparation agents can include, without limitation, a thickening agent, a gelling agent, a stabilizer, a water binder, and the like. In particular, the tWPC of the present invention can itself be used as a food preparation agent. One of ordinary skill in the art would readily understand how the tWPC can be used as a food preparation agent described herein.

In another aspect, the present invention relates to an emulsifier for use in food preparations, where the emulsifier includes a texturized whey protein composition of the present invention. In particular, the tWPC of the present invention can itself be used as an emulsifier in preparing foods in need of an emulsifier. One of ordinary skill in the art would readily understand how the tWPC can be used as an emulsifier described herein.

In another aspect, the present invention relates to an oil-in-water emulsion. In one embodiment, the oil-in-water emulsion includes (i) an aqueous phase containing a food emulsifier that includes a texturized whey protein composition of the present invention; and (ii) an oil phase dispersed in the aqueous phase. In one embodiment of the oil-in-water emulsion of the present invention, the texturized whey protein composition is present in an amount of between about 4 and about 16 weight percent, or more particularly at about 4, 5, 6, 7, 8, 9, 10, 11, 12, 13, 14, 15, or 16 weight percent (including degrees thereof as measured in 10ths or 100ths of a weight percent).

In another aspect, the present invention relates to a method of preparing an edible cold-setting gel-like emulsion. This method involves (i) providing an aqueous phase containing a texturized whey protein composition of the present invention; and (ii) dispersing an oil phase in the aqueous phase under conditions effective to yield an edible cold-setting gel-like emulsion. In one embodiment of this method, the texturized whey protein composition is provided in an amount of between about 4 and about 16 weight percent, or more particularly at about 4, 5, 6, 7, 8, 9, 10, 11, 12, 13, 14, 15, or 16 weight percent (including degrees thereof as measured in 10ths or 100ths of a weight percent). A suitable oil phase for use in this method can include any animal-derived oil or plant-derived oil. Examples of suitable animal-derived or plant-derived oils can include, without limitation, corn oil, canola oil, olive oil, almond oil, sunflower oil, safflower oil, peanut oil, palm oil, coconut oil, rice bran oil, soybean oil, rapeseed oil, cottonseed oil, palm kernel oil, sesame seed oil, lard, and the like. One of ordinary skill in the art would readily understand how to prepare an aqueous phase containing tWPC as the emulsifier, and then how to disperse an oil phase in the aqueous phase.

In another aspect, the present invention relates to a food product that includes an oil-in-water emulsion of the present invention. Examples of food products including the oil-in-water emulsion of the present invention can include, without limitation, mayonnaise, margarine, butter spreads, butter-like spreads, salad dressings, texturizing cream products, reduced fat formulated food products, whipping cream, and the like. Further, the food product can also include food formulations that contain heat-sensitive ingredients, particularly since the tWPC does not require the addition of heat energy to yield its enhanced functional properties. One of ordinary skill in the art would readily understand how to use the tWPC derived oil-in-water emulsion to prepare a food product containing the emulsion.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and described the methods and/or materials in connection with which the publications are cited.

It must be noted that as used herein and in the appended claims, the singular forms “a,” “and,” and “the” include plural references unless the context clearly dictates otherwise. All technical and scientific terms used herein have the same meaning

Publications discussed herein are provided solely for their disclosure prior to the filing date of the described application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Rheological Characterizations of Texturized Whey Protein Concentrate-Based Powders Produced By Reactive Supercritical Fluid Extrusion

Examples 1-3 correspond to various experiments performed to determine rheological characterizations of texturized whey protein concentrate-based powders produced by reactive supercritical fluid extrusion.

A powder blend comprising (by weight) 94% whey protein concentrate (WPC80), 6% pre-gelatinized corn starch, 0.6% CaCl₂, and 0.6% NaCl was texturized using a supercritical fluid extrusion (SCFX) process. The blend was extruded at 90° C. in a pH range of 2.89 to 8.16 with 1% (db) supercritical carbon dioxide (SC—CO₂) and 60% moisture content. The texturized WPC-based (tWPC) samples were dried, grounded into powder, reconstituted in water, and evaluated using a range of rheological studies. Most tWPC samples exhibited shear thinning behavior and their mechanical spectra were typical of weak gel characteristics. The tWPC produced under extremely acidic condition of pH 2.89 with SC—CO₂ yielded the highest η* (10,049 Pa s) and G′ (9,885 Pa) compared to the unprocessed WPC (η*=0.083 Pa s and G′=0.036 Pa). The SCFX process rendered WPC into a product with cold-setting gel characteristics that may be suitable for use as a food texturizer over a wide range of temperatures.

Rheological measurements have been considered as an analytical tool to provide fundamental insights on the structural organization of materials. The small-amplitude oscillatory (dynamic) tests (SAOS) have been commonly used to characterize the viscoelastic behaviors of food samples and also gel-like materials. It allows researchers to relate dynamic rheological parameters to the molecular structure of materials (Gunasekaran & Ak, 2000). The overall objective of this study was to determine the influence of altering pH and SC—CO₂ injection using reactive SCFX process on the rheological behaviors of tWPC powders upon reconstitution with water at ambient temperature. The performance of thickening and gelling of tWPC powders was evaluated using a range of rheological techniques. The ability of tWPC powders to impart instantaneous thickening and gelling functionality over a range of temperature was also investigated.

### Example 2

### Materials and Methods

### Materials and Feed Formulation

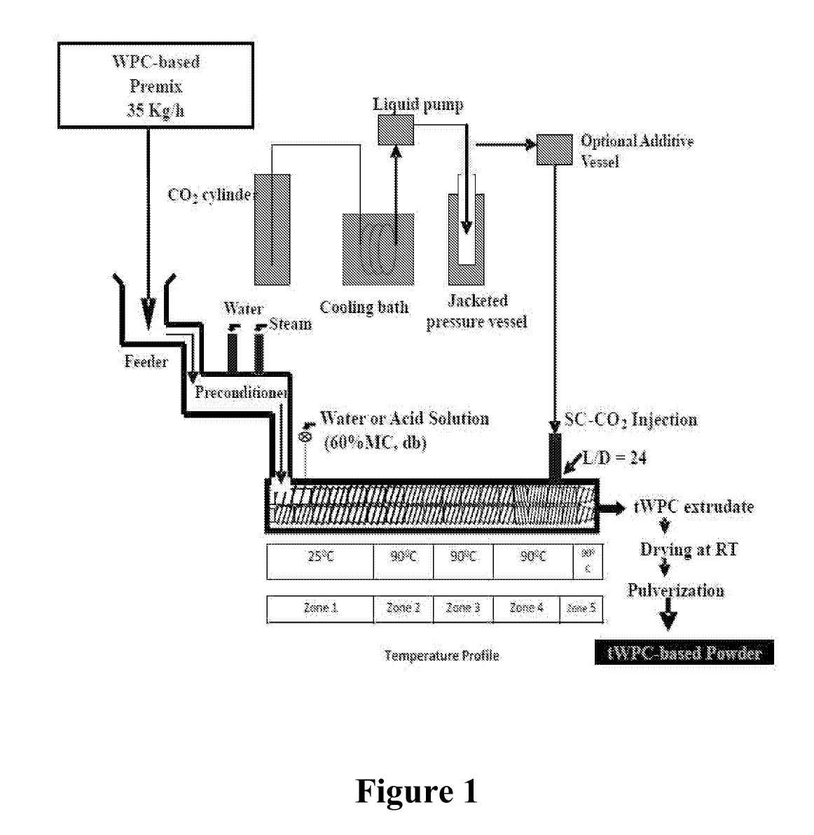

A commercial 80% (dry basis, db) WPC (lactalbumin-493) was obtained from Leprino Foods Company (Lemoore West, Calif., USA). The compositions of the WPC were 81.5% protein (db), 5.5% fat, 4% moisture, and less than 3% ash. Pre-gelatinized corn starch (Hammond, Ind., USA), NaCl, and CaCl₂ (Sigma Chemical Co., St. Louis, Mo., USA) were also added to a WPC-based dry mix. The pH-adjusting agents, NaOH and HCl solutions (Sigma Chemical Co., St. Louis, Mo., USA), were injected to the extruder at the mixing zone 1 (FIG. 1). Pre-hydrated (10% db) WPC80 powder (94%, w/w), pre-gelatinized corn starch (6%, w/w), CaCl₂ (0.6%, w/w), and NaCl (0.6%, w/w) were blended and then preconditioned at ambient temperature overnight before feeding into the extruder.

### Texturization of WPC by SCFX Process

A pilot-scale Wenger TX-52 Magnum (Wenger Manufacturing, Sabetha, Kans., USA) co-rotating twin screw extruder was used to texturize WPC-based blend. This extruder with 4.5 heads, a barrel diameter of 52 mm, and a length to diameter ratio (L/D) of 28.5:1 was specially configured for the process. The SCFX process was operated at the screw speed of 180 rpm, product temperature of 90° C., and feed rate of 35 kg/h. The die was fitted with two circular inserts of 1.2 mm diameter each. The screw configuration, and temperature control zones for extrusion are illustrated in FIG. 1. Extrusion was conducted at 60% (db) moisture content. To modify the pH of WP polymer melts in the extrusion process, HCl and NaOH solution streams at different concentrations were injected to the extruder at the mixing zone. The 15, 10, and 5% (v/v) HCl solutions were used to create the pH of 2.89, 3.53, and 4.44, respectively. In addition, the 0.83 and 1.67% (w/v) NaOH solution streams were injected to the extruder to create the pH of 7.01 and 8.16, respectively. These were compared with WPC-based blend extruded with deionized water stream only (non-pH-adjusted). A pilot scale supercritical fluid system was used for injecting SC—CO₂ into the protein polymer melts at L/D of 24 through four injection valves located around the extruder barrel. The die pressure was maintained higher than the pressure inside the barrel for continuous SC—CO₂ flow into the protein polymer melt, at the desired rate (1% db) and pressure (10-15 MPa). Product temperatures were monitored by a thermocouple at the end of the extruder. The extrudates were collected, dried at room temperature overnight or until their moisture contents were between 5 and 7%, and stored at room temperature in sealed containers.

### tWPC-Based Powder Preparation

Dried tWPC-based extrudates were grounded into powders using the mixer/mill machine (model 8000M-115 Mixer/Mill, SPEX CertiPrep, LLC, Metuchen, N.J., USA) to reduce the sample size to less than or equal to 125 μm. All samples were then stored at room temperature in air tight containers until analyzed.

### Rheological Characterizations

### Viscosity Measurement and Flow Behavior by Shear Rate Ramp Test

The tWPC and unprocessed WPC blend (control) powders were reconstituted at 20% (w/w) concentration in deionized water and gently stirred for 2 hours or until dissolution was completed, and then stored overnight at 4° C. prior to testing. This was done to ensure that dispersions were in the fully recovered state. The parallel plate geometry with 50 mm plate diameter was utilized for steady shear rate ramp test. The sample was loaded into the rheometer (ARES strain-controlled rheometer, TA Instruments, New Castle, Del., USA) equipped with a Peltier temperature controlling system, and then the top plate was slowly lowered until the final sample thickness of 1 mm was achieved. A thin layer of mineral oil was applied to the exposed sample edges to prevent the moisture loss. All experiments were conducted at 25° C. Shear rate was ramped from 1 to 100 s⁻¹. Shear stress (τ), shear rate (\dot{γ}), and apparent viscosity (ηₐ) were recorded by TA Orchestrator software. The corrected flow curves were fitted using the power law (Eq. 1) and Herschel-Bulkley model (Eq. 2). The flow behavior index (n), consistency coefficient (k), and yield stress (τ_{0HB}) were reported.

τ=k\dot{γ}ⁿ (1)

$${η}_{a} = \frac{{τ}_{OHB}}{\dot{γ}} + k {\dot{γ}}^{n - 1} \qquad ( 2 )$$

To investigate the effect of protein concentration, tWPC and unprocessed WPC dispersions were prepared at concentrations of 10%, 12.5%, 15%, 17.5% and 20% (w/w). These samples were allowed to hydrate overnight, and subjected to the same shear rate ramp test at 25° C. All determinations were done in triplicates.

### Viscoelastic Properties by Small-Amplitude Oscillatory (Dynamic) Tests

### Frequency Sweep Test

tWPC and unprocessed WPC blend (control) powders were reconstituted (30%, w/w) in deionized water and gently stirred for 2 hours at ambient temperature and allowed to hydrate overnight at 4° C. Viscoelastic properties of all samples were monitored using the rheometer (ARES strain-controlled rheometer, TA Instruments, New Castle, Del., USA) equipped with a Peltier temperature controlling system, and utilizing a parallel plate geometry (25 mm plate diameter). The sample was loaded into the rheometer, and then the top plate was slowly lowered until the final sample thickness of 2 mm was achieved. A thin layer of mineral oil was applied to the exposed sample edges to prevent moisture loss. The frequency was oscillated from 0.1 to 100 rad/s at 25° C. All measurements were performed within the identified linear viscoelastic region and made at 1% strain. The storage modulus (G″), loss modulus (G″), complex viscosity (η*), and loss angle tangent (tan δ) were then recorded by TA Orchestrator software. All determinations were done in triplicates

### Temperature Sweep Test

The thermal stability of selected 20% (w/w) tWPC and unprocessed WPC dispersions were monitored using the same rheometer and procedure with 25 mm diameter parallel plate geometry, and 2 mm sample thickness. The temperature was ramped from 25° C. to 85° C. at 2° C./min heating rate and at a constant frequency rate of 1 rad/s and 1% strain. The storage modulus (G″), loss modulus (G″), complex viscosity (η*), and loss angle tangent (tan δ) were then recorded by TA Orchestrator software. All determinations were done in triplicates

### Water Holding Capacity (WHC) of tWPC Powders

The selected tWPC powders were hydrated (15%, w/w) in deionized water for 3 hours and centrifuged at 3,500 RPM for 30 min at 25° C. After centrifugation, the supernatant was removed and the remaining pellet was weighed. The amount of water held per gram of protein powder was calculated as the WHC. All determinations were done in triplicates

### Statistical Analysis

Statistical analysis was done using MINITAB® release 14 statistical software (State College, Pa., USA). Significant differences (p<0.05) were determined by analysis of variance using the general linear models and least square means procedure.

### Example 3

### Results and Discussion

### General Observation on tWPC Production

The 6% (w/w) pre-gelatinized corn starch was added to the feed formulation for the extrusion process in order to facilitate the manufacturing of tWPC extrudates. The preliminary studies revealed that the extrusion process of 100% WPC powder was difficult to achieve. The pre-gelatinized corn starch has been used as a binder to hold protein matrices because of their ability to form hydrogen bonds in the extruded products (Amaya-Llano, Morales Hernandez, Castaño Tostado, & Martinez-Bustos, 2007). Considerably, in this process the pre-gelatinized corn starch acted as an inactive filler in the tWPC extrudate formation. According to Aguilera and Rojas (1996) the rheological properties of heat-induced WP gels were significantly influenced when 10 to 20% (w/w) of WP in the system was substituted by corn starch. However, a treatment with WPC only was preliminarily investigated in order to assess the effects of starch on the final gel characteristics of the extruded WPC samples. It showed that the addition of 6% (w/w) pre-gelatinization corn starch to the feed formulation did not have a significant effect on the rheological characteristics of final tWPC products due to the apparently low starch content (0.6 to 1.8%, w/w) in the studied WP dispersions. Therefore, the interaction between starch and protein would be insignificant, given that the structure of the extrudates was dominated by protein-protein interactions during extrusion. The main factor in this study that could influence and dominate the structural changes in WP was extrusion pH. Hudson and Daubert (2002), and Shim and Mulvaney (2001) indicated that changes in WP structure were significantly dependent on pH while starch was relatively insensitive to pH changes.

The extrusion process was more severe in alkaline than in acidic conditions. Onwulata et al. (2006) reported similar observations during extrusion of WPI under alkaline conditions. A stronger alkaline treatment produced the WP extrudate with a dry rough surface. Based on the visual observation, alkaline conditions increased the dark yellow color and the toughness of the extrudates. The acidic conditions, on the other hand, resulted in transparent and light yellow color extrudates with a smooth surface, which fractured more ease after the drying process.

Injection of 1% (db) SC—CO₂ to the pH-treated tWPC slightly reduced the pH of the final products. The incorporation of SC—CO₂ into protein polymer melts in the extruder was achieved by injecting it above its critical conditions (T_{c}=31° C. and P_{c}=7.38 MPa) (Chen, 2005). In supercritical phase, SC—CO₂ is readily dissolved into the water phase in the protein polymer melts, and forms carbonic acid providing additional acidity to final tWPC products. Injection of SC—CO₂ to the pH-treated WPC melts produced a lighter product color visually apparent in most extrudate samples.

### Apparent Viscosity

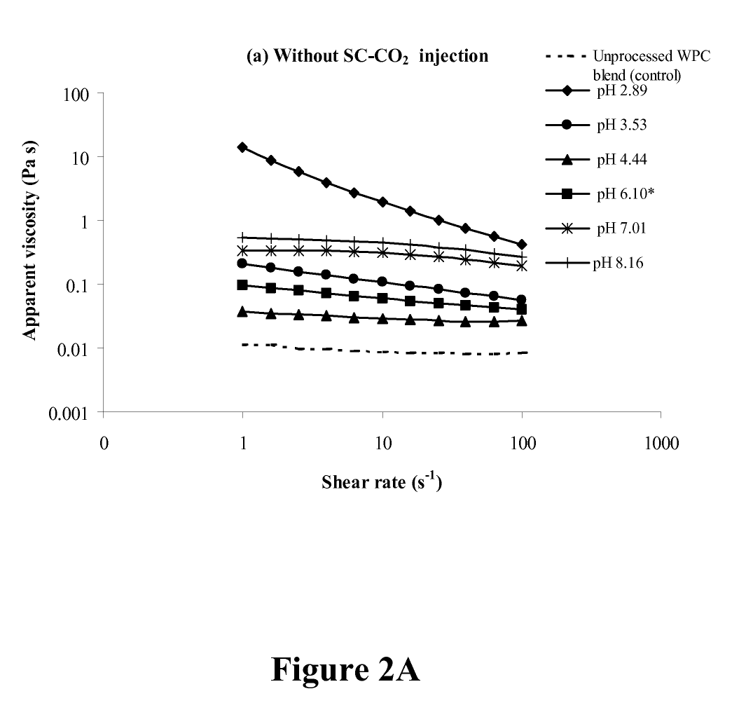

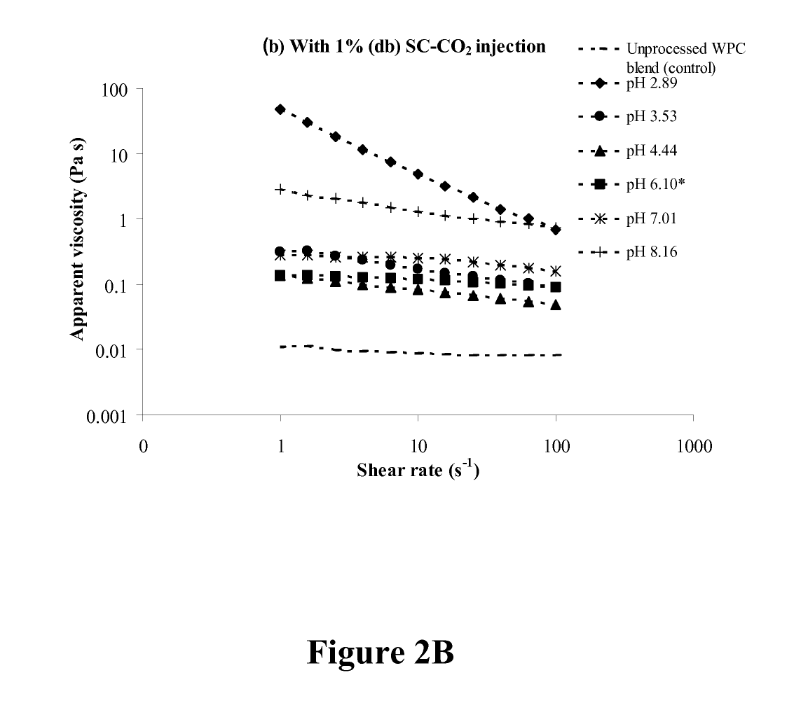

The variation in the apparent viscosity of dispersions containing 20% (w/w) tWPC powders produced at different pH compared with the unprocessed WPC blend is illustrated in FIG. 2A. The viscosity of most tWPC samples was shear rate dependence, except for the tWPC produced at pH 4.44 which appeared to be Newtonian similar to that of the unprocessed WPC. Data revealed in Table 1 indicated that the acid-treated tWPC had apparent viscosities at 25 s⁻¹ shear rate (η₂₅) ranging from 0.026 to 0.988 Pa s while the alkali-treated tWPC had the η₂₅ of 0.292 to 0.444 Pa s. The non-pH treated tWPC had the average η₂₅ of 0.052 Pa s while the unprocessed WPC blend showed the lowest η₂₅ of 0.008 Pa s. The enhanced apparent viscosity was shown in most of the pH-treated tWPC samples. This indicated an increase in unfolding and denaturation of globular proteins when the pH of WPC was altered. In general, unfolding of globular proteins is frequently accompanied by an increase in the hydrodynamic radii of protein molecules and greater molecular entanglements which contribute to an increase in the apparent viscosity of WP solutions (Rattray & Jelen, 1995). This finding suggests that adjusting acidity (H⁺) and alkalinity (OH⁻) of WPs while heating and shearing during extrusion could induce the conformational structure alteration of proteins since their net charges have been manipulated. According to Harding (1998), proteins are all polyelectrolytes and possess electrostatic charge which can affect the viscosity. This electrostatic contribution is strongly dependent on the pH and the ionic strength of solution. As pH was adjusted away from the isoelectric point (pH 5.0-5.3), the viscosity of WP solutions significantly increased (Rector, 1992).

TABLE 1
Rheological parameters^{a} of models describing flow behaviors of dispersions containing
20% (w/w) tWPC and unprocessed WPC blend powders

| | η₂₅ | Power law model | | | Herschel-Bulkley model | | | |
|---|---|---|---|---|---|---|---|---|
| Extrusion pH | [Pa s] | k [Pa sⁿ] | n | R² | τ_{0HB} [Pa] | k [Pa sⁿ] | n | R² |
| Unprocessed WPC | 0.008 | 0.011 | 0.928 | 0.999 | 0.000 | 0.009 | 0.989 | 0.999 |
| blend (control) | | | | | | | | |
| Without SC-CO₂ | | | | | | | | |
| 2.89 | 0.988 | 11.731 | 0.250 | 0.953 | 10.820 | 2.290 | 0.560 | 0.999 |
| 3.53 | 0.083 | 0.203 | 0.720 | 1.000 | 0.010 | 0.196 | 0.733 | 1.000 |
| 4.44 | 0.026 | 0.035 | 0.922 | 0.999 | 0.010 | 0.028 | 0.969 | 0.999 |
| 6.10^{b} | 0.052 | 0.095 | 0.807 | 0.999 | 0.022 | 0.082 | 0.855 | 0.999 |
| 7.01 | 0.292 | 0.370 | 0.883 | 0.997 | 0.400 | 0.638 | 0.740 | 0.999 |
| 8.16 | 0.442 | 0.579 | 0.855 | 0.998 | 0.800 | 1.022 | 0.716 | 0.999 |
| With 1% (db) SC-CO₂ | | | | | | | | |
| 2.89 | 2.063 | 43.028 | 0.078 | 0.771 | 42.480 | 1.500 | 0.560 | 0.985 |
| 3.53 | 0.122 | 0.338 | 0.711 | 0.999 | 0.118 | 0.246 | 0.770 | 0.999 |
| 4.44 | 0.068 | 0.136 | 0.778 | 1.000 | 0.005 | 0.133 | 0.789 | 0.999 |
| 6.10^{b} | 0.025 | 0.144 | 0.906 | 0.999 | 0.001 | 0.026 | 0.990 | 0.999 |
| 7.01 | 0.249 | 0.303 | 0.884 | 0.998 | 0.400 | 0.538 | 0.740 | 0.999 |
| 8.16 | 1.031 | 2.650 | 0.710 | 0.998 | 1.618 | 2.271 | 0.750 | 0.998 |

^{a}Mean of triplicates; η₂₅ - apparent viscosity at 25 s⁻¹ shear rate; k - consistency coefficient; n - flow behavior index; τ_{0HB} - yield stress.
^{b}tWPC powder extruded with water only (non-pH-adjusted).

The tWPC produced at extremely acidic condition of pH 2.89 yielded the highest apparent viscosity while the products at pH 3.53 and 4.44 evidently exhibited much lower apparent viscosities at any given shear rate. At pH 2.89, the apparent viscosity of WPC has been ultimately improved by approximately 124 times (η₂₅=0.988 Pa s) compared to the unprocessed WPC sample (η₂₅=0.008 Pa s) (Table 1). It was also observed that at 20% (w/w) WP dispersion, tWPC produced at pH 2.89 yielded a highly homogeneous, smooth, and viscous dispersion texture. It is interesting to note that an increase in apparent viscosity of highly acid-treated tWPC is the evitable outcome of acid-induced protein denaturation (Rattray & Jelen, 1995). The denaturation of α-lacalbumin (α-La) and bovine serum albumin (BSA) at pH 3.5 or lower were documented (Bernal & Jelen, 1894; Boye, Kalab, Alli, & Ma, 2000). However, β-lactoglobulin (β-Lg), the most abundant protein in whey (˜50% of total WP), is more rigid and resistant to extensive denaturation at acid pH (pH<3.5) (de Wit & Klarenbeek, 1984; Jelen & Buchheim, 1984; Monahan, German, & Kinsella, 1995; Taulier & Chalikian, 2001). As described earlier, heating and shearing in the extruder led to protein unfolding, thus rendering the proteins more susceptible to further denaturation by acid treatment. Perhaps by thermal and mechanical energy provided by extrusion process, β-Lg could be more denatured and contributed to high apparent viscosity or gelation at low pH. Accordingly, Rattray and Jelen (1995) reported that thermal treatment of acidic WPC solutions (11% and 20%, w/v, protein) at pH values <3.5 caused relatively strong gels due to protein denaturation and aggregation. A similar observation was also reported by Singer, Yamamoto, and Latella (1988). The authors demonstrated that a highly viscous, semi-solid product with lipid-like texture could be produced by simultaneously heating and shearing of liquid WPC process at acid pH.

It is evident that increasing pH of WPC to 8.16 resulted in a moderate increase in apparent viscosity (Table 1). As reported by Monahan et al. (1995), at alkali pH, proteins attain an overall negative charge groups. If enough negative charge is present, adjacent portions of the protein will start to repel each other due to the high charge density. When this occurs, the protein begins to unfold and the molecules' radius of gyration should expand and result in an increased viscosity. Consequently, the exposure of sulfhydryl (SH—) group and thiol-disulfide interchange are expected to occur which leads to a higher rate of aggregation and denaturation (Watanabe & Klostermeyer, 1976). Therefore, extruding WPC in highly alkaline conditions could produce larger aggregates and/or high molecular weight denatured proteins. The tWPC produced at extremely alkaline condition (pH 8.16) exhibited a coarse and gritty dispersion texture due to the higher extent of insoluble large aggregated proteins contained in the sample. This was also observed in our SDS-PAGE electrophoresis studies. However, WPs with a higher degree of aggregation were found to have increased viscosity due to the aggregates greater effective volume which could entrap more water than the individual WP molecules (Firebaugh, 2004). These findings are similar to those of texturized soy protein studied by Dahl and Villota (1991) and Fleming, Sosulski, Kilara, and Humbert (1974). Their results indicated that the viscosity of soy proteins increased markedly after proteins were modified by the alkaline treatment.

The combined effects of SC—CO₂ and pH treatment on the apparent viscosity of tWPC dispersions are illustrated in FIG. 2B. It is clearly shown that injecting 1% (db) SC—CO₂ into pH-adjusted WP polymer melts generally improved the apparent viscosity of final tWPC products at comparable shear rate. As shown in Table 1, incorporation of SC—CO₂ noticeably improved the viscosity of tWPC samples extruded at pH 2.89, 3.53, 4.44 and 8.16. Considerably, the effect of SC—CO₂ addition on the viscosity of tWPC samples became more pronounce when WPC blend was extruded at extremely acidic (pH 2.89) or alkaline (pH 8.16) conditions. At extreme acid condition, the apparent viscosity (η₂₅) of tWPC dispersion significantly (p<0.05) increased from 0.988 Pa s to 2.063 Pa s with SC—CO₂ addition. For extreme alkali condition, the η₂₅ of tWPC dispersion significantly (p<0.05) increased from 0.442 Pa s to 1.031 Pa s with SC—CO₂ addition. Overall, incorporation of 1% (db) SC—CO₂ contributed approximately 258 and 129 times higher viscosity in tWPC produced at pH 2.89 and 8.16, respectively, compared to the unprocessed WPC sample (η₂₅=0.008 Pa s). Results revealed that tWPC produced under extremely acidic condition of pH 2.89 with SC—CO₂ treatment yielded the maximum apparent viscosity at equal shear rate. Combination of highly acid environment and SC—CO₂ addition by SCFX process possibly influenced hydrophobic interactions versus non-covalent linkages, such as intermolecular hydrogen bonding and electrostatic interactions. These interactions are found to be important in hydrocolloids to function as thickening agents in some food products such as salad dressing and sauces (Lang & Rha, 1981).

### Flow Behavior

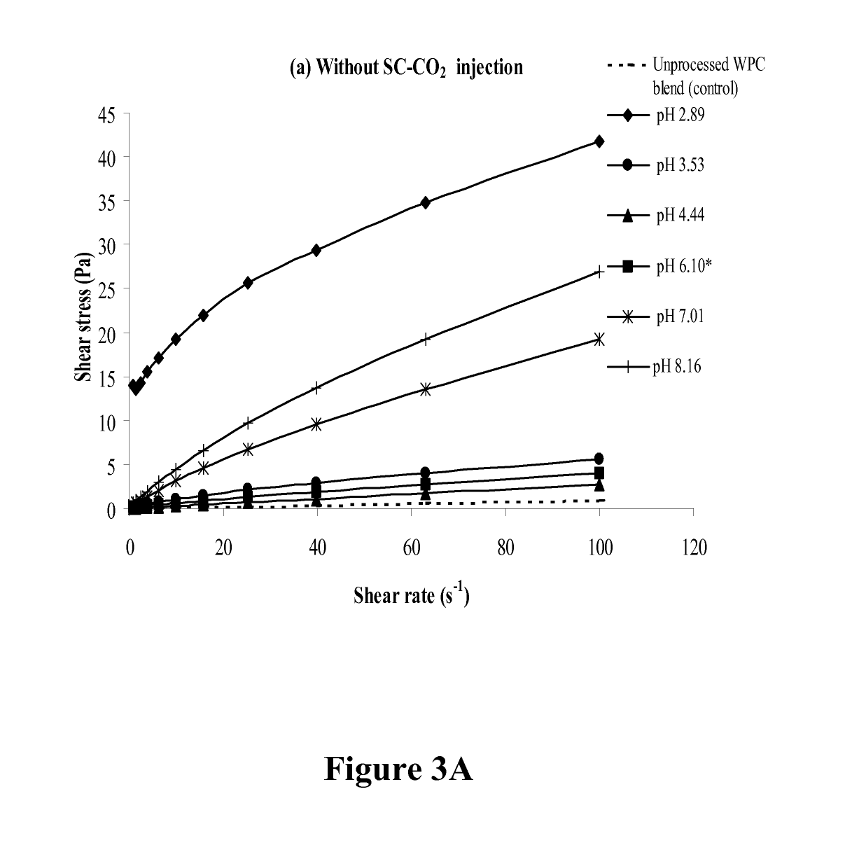

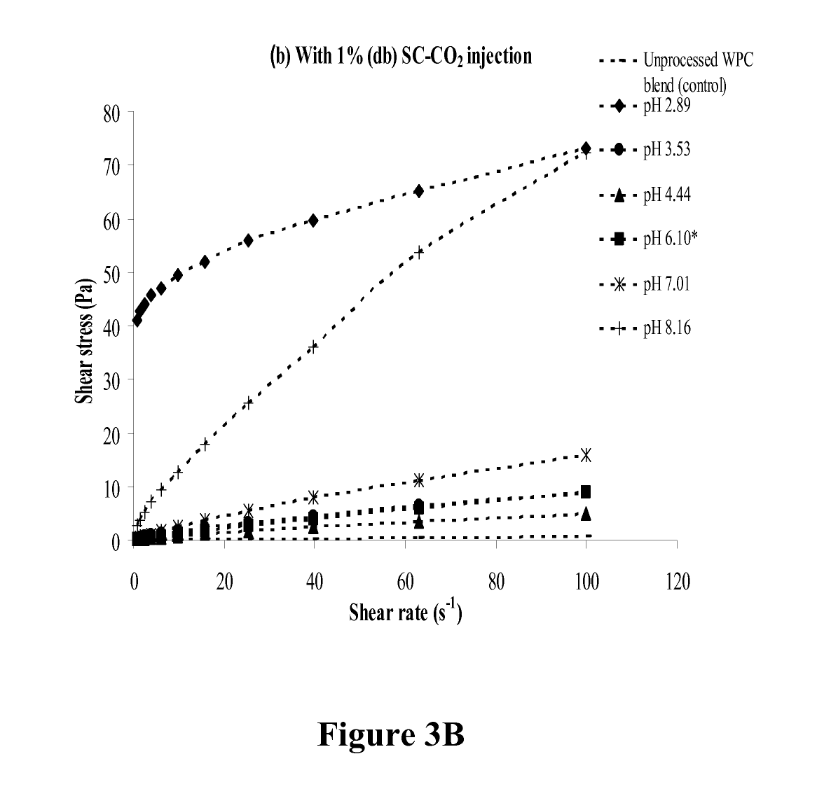

The decreasing viscosity when shear rate is increasing observed in most tWPC samples implies the pseudoplastic flow behavior or shear thinning behavior which is commonly seen in food thickeners (Daubert, Resch, & Foegeding, 2004). To study their flow behaviors, shear stress-shear rate data of dispersions (20% protein concentration, w/w) of pH-treated tWPC with and without SC—CO₂ injection (FIGS. 3A and 3B) and the unprocessed WPC were fitted to two different rheological models, the power law and Herschel-Bulkley. The power law model does not take account of yield stress. On the other hand, the Herschel-Bulkley model contains yield stress (τ_{0HB}) which is a very important rheological parameter and has been often used to explain the flow behavior of pseudoplastic material properties (Curran, Hayes, Afacan, Williams, & Tanguy, 2002; Hudson & Daubert, 2002). The yield stress (τ_{0HB}), consistency coefficient (k), and flow behavior index (n) of tWPC dispersions compared to the unprocessed WPC were summarized in Table 1. The coefficients of determination (R²) for each sample were always greater than 0.95, supporting the validity of the selected models. Although the flow behaviors of tWPC dispersions were represented well by both models, the Herschel-Bulkley model exhibited a better fit in most samples regarding the higher R² values. This occurred because most tWPC samples displayed the yield stress values as shown in Table 1.

The flow behavior index (n) values were generally greater for the Herschel-Bulkley model than for the power law model. This was probably due to the different fit of the two models to the experimental data and to the fact that the power law model disregards yield stress. However, the n values from both models were less than 1.0, implying the pseudoplasticity of most tWPC samples. Nonetheless, the tWPC powder produced at pH 4.44 and the unprocessed WPC exhibited Newtonian behavior confirmed by their n values which approached 1.0. Results depicted in Table 1 show that the n values from the Herschel-Bulkley model for pH-treated tWPC with injected SC—CO₂ is in the range of 0.56 to 0.78. This indicates the shear-thinning or pseudoplastic nature of particulate aggregate or weak gel type of flow (Rector, 1992). The tWPC treated with extreme acid (pH 2.89) and SC—CO₂ showed the greatest values of shear stresses in a given range of shear rates with markedly highest yield stress (FIG. 3B). It also had the highest consistency coefficient and lowest flow behavior index based on the power law model (Table 1). This result is also confirmed by the steepest viscosity rise at low shear rate presented in FIG. 2B. It can be attributed to the breakdown of the inner structure of fluid which is formed through physical interactions between molecules (Gerhards & Schubert, 1993). As the shear rate increases, those forces weaken and the molecules orient themselves along the flow lines, which causes a drop in viscosity. The higher yield stress represents the stronger gel network in which higher stress is required to break a gel and initiate flow. Hudson and Daubert (2002) reported that the yield stress appeared in derivatized WPI was due to hydrophobic interactions versus non-covalent linkages. Such result would suggest that SCFX process rendered WPC into a product with thickening characteristics.

### Influence of Protein Concentration

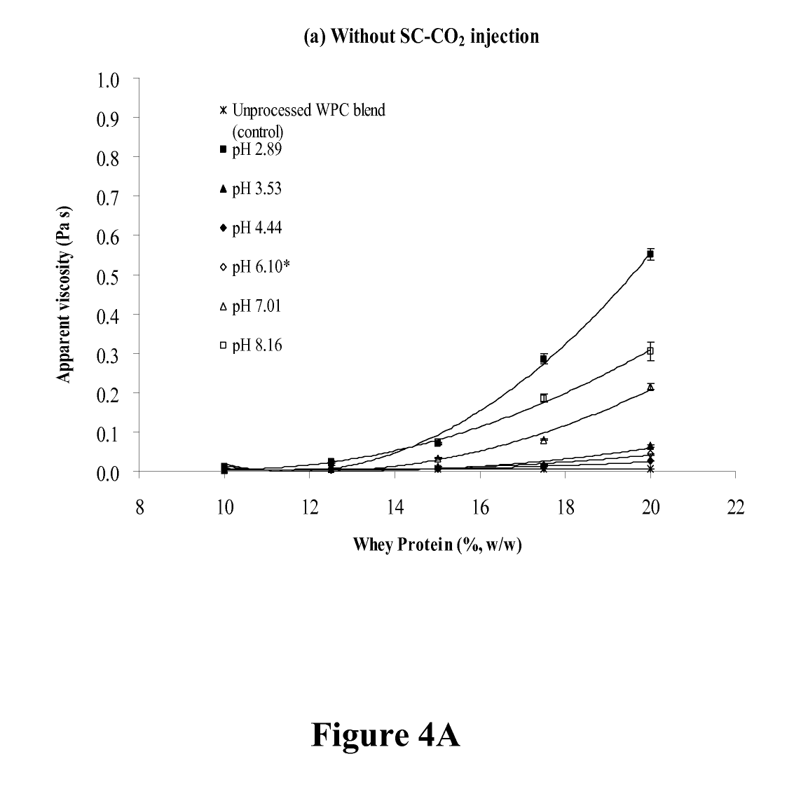

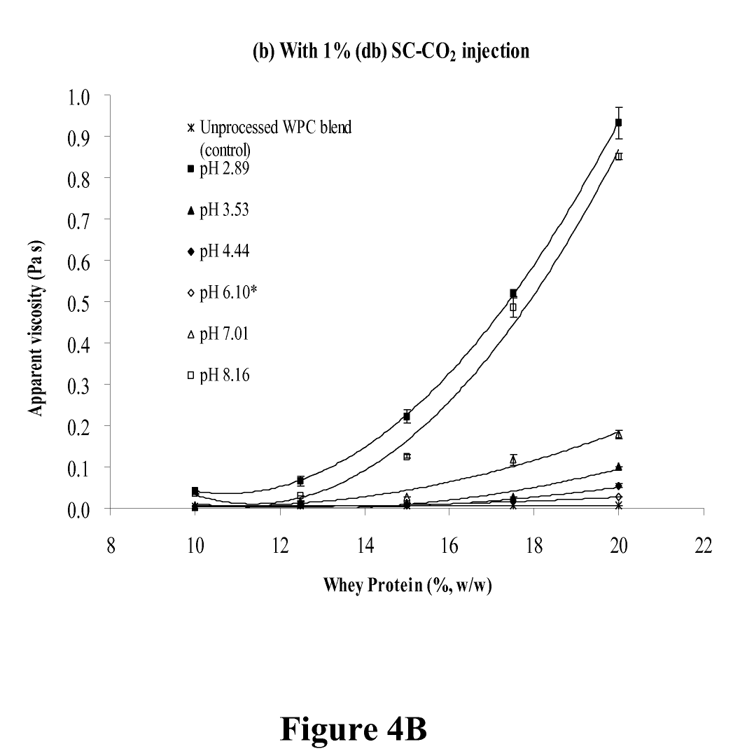

The effect of protein concentrations (10, 12.5, 15, 17.5, and 20%, w/w) on the apparent viscosity at 61.15 s⁻¹ shear rate (η_{61.15}) of tWPC samples with and without SC—CO₂ treatment is illustrated in FIGS. 4A and 4B. Results revealed that all tWPC samples exhibited the ability to impart a wide range of viscosities by varying protein concentrations. It clearly shows that the apparent viscosity of most samples increased with the increase in protein concentration. With 1% (db) SC—CO₂ injection, the greater apparent viscosity of tWPC samples was observed in all concentrations. Drastic increase in apparent viscosity at higher protein concentrations was observed in tWPC samples produced at pH 2.89 and 8.16 with SC—CO₂ treatment. Notably, the tWPC produced at pH 2.89 with SC—CO₂ noticeably yielded the highest apparent viscosity at equal concentration, and its apparent viscosity increased by 22 times when the concentration of protein increased from 10 to 20% (w/w). On the other hand, the protein concentration did not largely influence the apparent viscosity of unprocessed WPC sample. Clark (1998) reported that the native WP will not usually achieve a high viscosity, even at moderately high concentrations, because of its folded globular shape. However, when WPs were more denatured, the increase in viscosity became more pronounced as generally observed in several tWPC samples. At higher protein concentrations, it was likely that denatured proteins were packed closely together, promoting protein-protein interactions, greater protein molecular entanglements, and polymerization, all of which contributed to the viscosity increases (Rector, 1992). In addition, swelling caused by hydrogen bonds between amino acid groups and water, resulting in an increase in the molecular radii of protein molecules could considerably be involved in the viscosity increase as well (Clark, 1998; Rattray & Jelen, 1995; Schmidt, Packard, & Morris, 1984). Thus, stabilizing and strengthening characteristics shown in of tWPC dispersions were more pronounced as the amount of denatured proteins increased.

### Viscoelastic Properties

In this study, the dynamic mechanical testing approach was used to measure mechanical changes in linear viscoelastic behavior of tWPC samples containing 30% (w/w) WP. Unlike the viscosity measurement, this small strain measurement is believed to leave microstructure intact and thus be able to characterize viscoelastic properties of the original fluid structure (Gunasekaran & Ak, 2000). A viscoelastic network indicates the elastic and viscous behavior of the sample over a range of frequencies. The storage modulus (G′) represents a measure of elastic response of the material whilst the loss modulus (G″) is a measure of the viscous response.

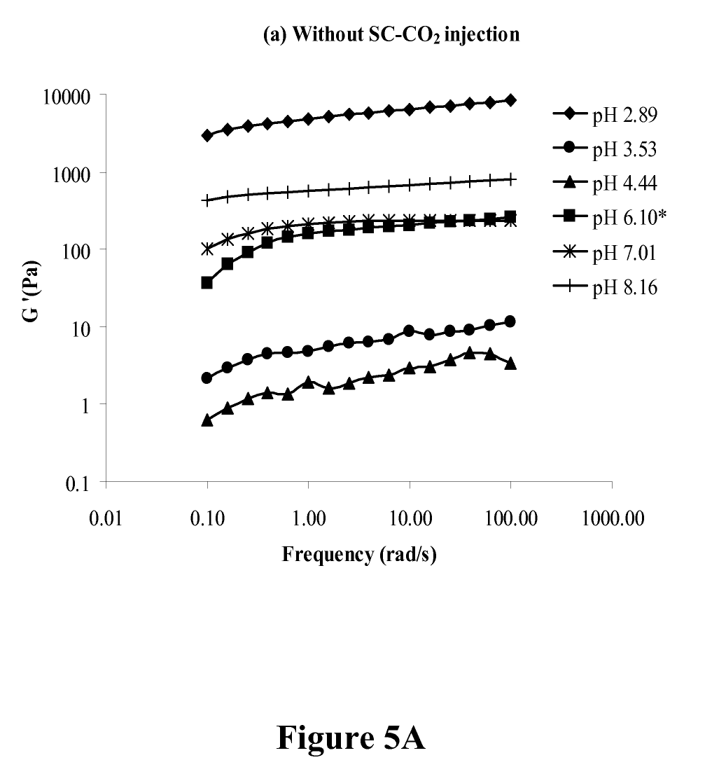

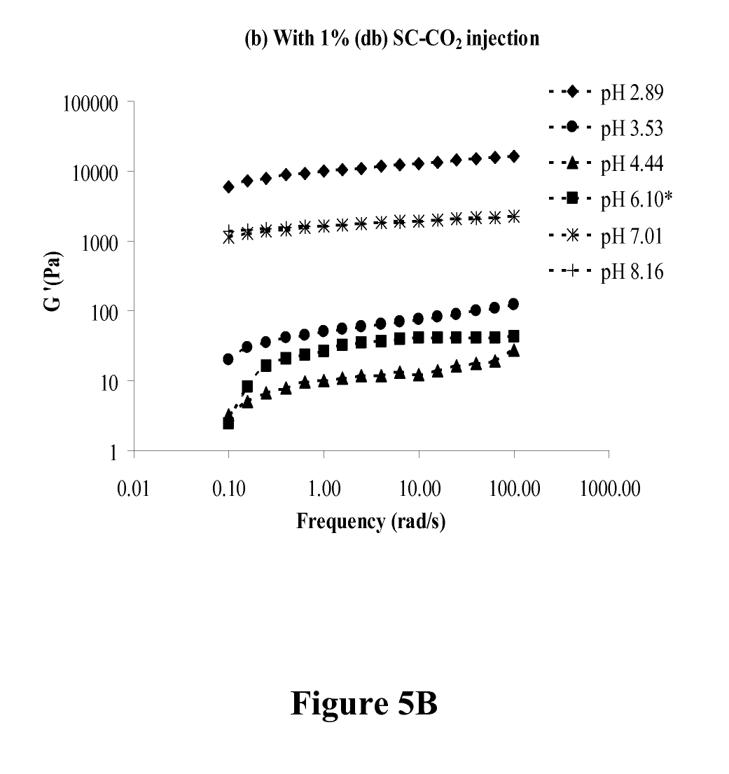

The unprocessed WPC data have not been included in the viscoelastic results due to its very poor consistency which prohibited measurement of its dynamic properties in the linear viscoelastic range. It did not show viscoelastic behavior as observed from its very low viscosity (G″>>G′) and it was impossible to obtain the plateau modulus. Results illustrated in FIG. 5A (without SC—CO₂) and FIG. 5B (with SC—CO₂) revealed that G′ of all tWPC samples increased as the frequency increased from 0.1 to 100 rad/s. The higher G′ than G″ over a range of frequency has been found in all tWPC samples (data not shown for G″). This testifies to the dominance of elastic properties over viscous ones. The similar characteristics have been also reported for starch and protein gels and mixed starch/WP gels (Beveridge & Timbers, 1985; Carvalho, Onwulata, & Tomasula, 2007; El-Garawany & Abd El Salam, 2005). The authors described that this behavior could be attributed to the cross-linkage formations by disulfide bonds and hydrophobic interactions in the gel structures. The lowest pH (2.89) and highest pH (8.16) tWPC samples had excessively large G′, while pH 6.10 and 7.01 samples had the moderate G′, and pH 3.53 and 4.44 samples showed the lowest G′ over a range of frequencies (FIGS. 5A-5B).

The elastic modulus (G′), loss modulus (G″), tan δ, and complex viscosity (η*) of tWPC and unprocessed WPC samples were compared as shown in Table 2. The frequency of oscillation chosen for comparison was 1 rad/s, which represents a timescale sufficiently short that even physical gel cross-links are effectively permanent. It was likely that by adjusting pH, the viscoelastic properties of WPC blend were manipulated, especially at highly alkaline and acid environments. Extruding WPC blend at extremely acidic concentration (pH 2.89) significantly (p<0.05) yielded the highest G′ (4798.43 Pa), G″ (975.41 Pa), and η* (4896.62 Pa s), followed by the highly alkaline treatment of pH 8.16 (G′=570.59 Pa, G″=111.08 Pa, and η*=581.33 Pa s). The moderate G′, G″, and η* were observed in pH 6.10 and 7.01 samples, while the pH 3.53 and 4.44 samples possessed the lowest G′, G″, and η* values.

TABLE 2
Storage modulus (G′), loss modulus (G″), tan δ, and complex
viscosity (η*) at the frequency of 1 rad/s of dispersions containing
30% (w/w) tWPC and unprocessed WPC blend powders^{a}

| Extrusion pH | G′ [Pa] | G″ [Pa] | tan δ | η* [Pa s] |
|---|---|---|---|---|
| Unprocessed WPC blend | 0.036^{a} | 0.075^{a} | 2.083^{a} | 0.083^{a} |
| (control) | | | | |
| Without SC—CO₂ | | | | |
| 2.89 | 4798.435^{e} | 975.410^{e} | 0.203^{g} | 4896.622^{e} |
| 3.53 | 4.327^{a} | 2.363^{a} | 0.546^{b} | 5.250^{a} |
| 4.44 | 1.895^{a} | 1.044^{a} | 0.550^{b} | 2.168^{a} |
| 6.10b | 156.853^{b} | 52.484^{b} | 0.335^{e} | 165.269^{b} |
| 7.01 | 210.755^{b} | 64.371^{b} | 0.305^{ef} | 220.384^{b} |
| 8.16 | 570.591^{c} | 111.081^{bc} | 0.195^{h} | 581.333^{c} |
| With 1% (db) SC—CO₂ | | | | |
| 2.89 | 9885.870^{f} | 1805.123^{f} | 0.183^{gh} | 10049.755^{f} |
| 3.53 | 50.169^{a} | 16.576^{a} | 0.330^{e} | 52.828^{a} |
| 4.44 | 9.966^{a} | 4.695^{a} | 0.479^{c} | 11.017^{a} |
| 6.10^{b} | 26.663^{a} | 10.538^{a} | 0.395^{d} | 28.679^{a} |
| 7.01 | 1617.065^{d} | 358.865^{d} | 0.221^{f} | 1656.413^{d} |
| 8.16 | 1651.812^{d} | 314.813^{d} | 0.196^{g} | 1681.645^{d} |

^{a}Means with the same superscript letter within a column are not significantly different (p < 0.05).
^{b}tWPC powder extruded with water only (non-pH-adjusted).

SCFX process with 1% (db) SC—CO₂ injection is shown to improve the viscoelastic properties of most pH-treated tWPC samples. The most pronounced changes in viscoelastic properties when the SC—CO₂ was incorporated were found in alkali-treated tWPC at pH 7.01, where G′, G″, and η* increased by approximately 8, 6, and 8 times, respectively (Table 2). However, injection of SC—CO₂ to the extreme acid-treated tWPC significantly (p<0.05) yielded the highest G′ (9885.87 Pa), G″ (1805.12 Pa), and η* (10049.75 Pa s), compared to the unprocessed WPC (G′=0.036 Pa, G″=0.075 Pa, and η=0.083 Pa s). It is clearly shown that extruding WPC blend at pH 2.89 with SC—CO₂ injection ultimately increased its G′, G″, and η* by approximately 274,000, 24,000, and 120,000 times, respectively, compared to the unprocessed WPC.

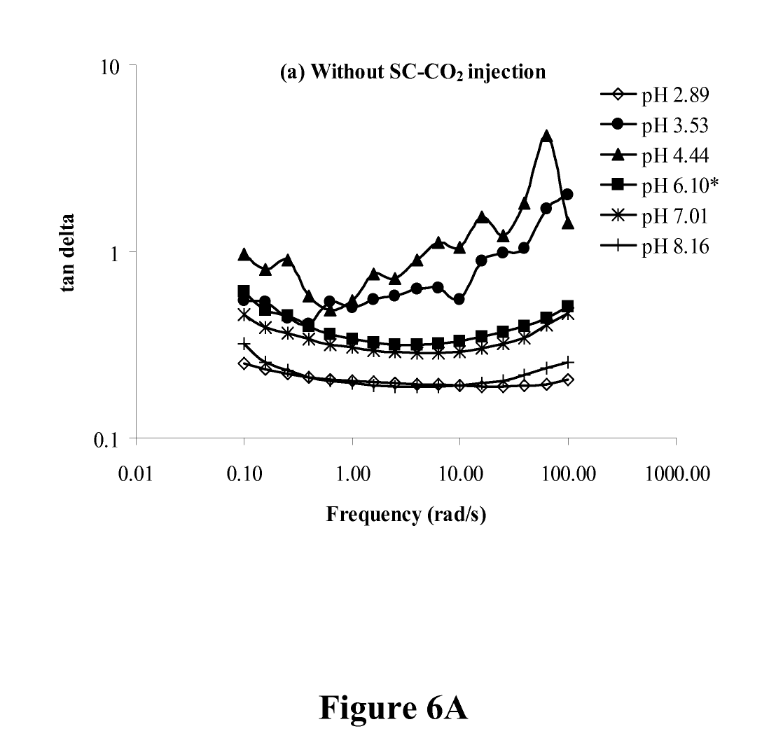

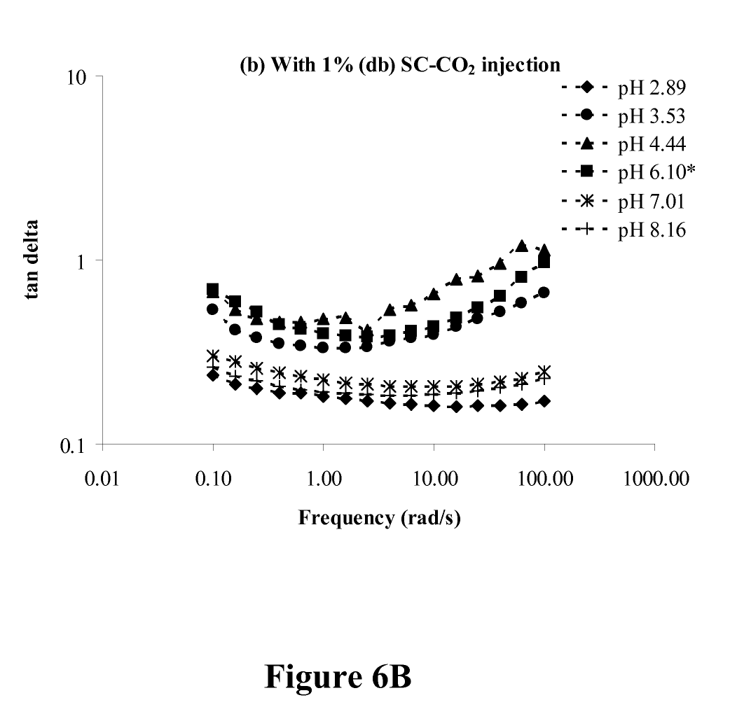

The relative ‘strength’ of gels could be interpreted in terms of tan δ(G″/G′), measuring energy loss compared to energy stored in cyclic deformation. It was indicated that gels with tan δ>0.1 had a paste-like quality (weak gel), while gels with tan δ<0.1 were firm, self-standing (true) gels (Clark, 1998; Shim & Mulvaney, 2001). In this study, tWPC samples showed a broader range of tan δ compared at the same frequency of 1 rad/s (0.183 to 0.550) which is greater than 0.1, indicating a weak gel or paste-like structure. A relative comparison of tan δ was shown over the entire range of frequencies for pH-treated tWPC without SC—CO₂ (FIG. 6A) and with 1% (db) SC—CO₂ injection (FIG. 6B). With SC—CO₂ addition, most tWPC samples showed a lower extent of tan δ over a range of frequencies, indicating the more solid gel behaviors, except for the non-pH adjusted tWPC (pH 6.10) sample. The almost linear relationship between tan δ and the applied frequencies of those samples also indicates the stronger gel networks and more solid-like behaviors. FIG. 6B and Table 2 reveal that the tWPC produced at pH 2.89 with SC—CO₂ injection significantly (p<0.05) yielded the strongest gel network indicated by the lowest tan δ (0.183).

It was reported that the absolute difference between G′ and G″ over a range of frequencies and the degree of frequency independency of moduli demonstrated the typical characteristics of gels (Clark, 1998; Rodd, Davis, Dunstan, Forrest, & Boger, 2000; Shim & Mulvaney, 2001). Table 3 shows the frequency dependence of the moduli (G′ and G″) analyzed quantitatively by fitting simple power law relationships;

G′∝ω^{p} (3)

G″∝ω^{q} (4)

where ω is the frequency of oscillation and p and q are the storage and loss moduli power law indices, respectively. The values of p and q, determined in this study, showed a slightly wide range of 0.069 to 0.279 for p, and 0.052 to 0.445 for q. The results shown in Table 3 confirm that accompanying SC—CO₂ with pH alteration using SCFX process produced a stronger gel network in most tWPC samples indicated by lower extent of frequency dependence of moduli (lower p and q values). Results indicated that the tWPC produced at pH 2.89, 7.01 and 8.16 with SC—CO₂ had a noticeably lowest frequency dependence of G′ and G″, implying the stronger internal structure compared to the rest of the samples. However, tWPC samples did not resemble those of solid gel characteristics regarding their frequency dependence of mechanical spectra (G′, G″, and tan δ) as previously presented. In addition, the differences between G′ and G″ values of all samples were less than one order of magnitude, indicating a weak gel entanglement network (Clark & Ross-Murphy, 1987). Among the samples studied, tWPC produced at pH 2.89 with SC—CO₂ had the strongest gel structure.

TABLE 3
Power law parameters^{a} describing the frequency
dependence of moduli (G′ and G″) of dispersions
containing 30% (w/w) tWPC powders

| Extrusion pH | p | q |
|---|---|---|
| Unprocessed WPC blend | —^{b} | — |
| (control) | | |
| Without SC—CO₂ | | |
| 2.89 | 0.138 | 0.109 |
| 3.53 | 0.208 | 0.380 |
| 4.44 | 0.252 | 0.445 |
| 6.10^{c} | 0.218 | 0.192 |
| 7.01 | 0.091 | 0.087 |
| 8.16 | 0.089 | 0.062 |
| With 1% (db) SC—CO₂ | | |
| 2.89 | 0.129 | 0.085 |
| 3.53 | 0.221 | 0.271 |
| 4.44 | 0.226 | 0.445 |
| 6.10^{c} | 0.279 | 0.324 |
| 7.01 | 0.092 | 0.059 |
| 8.16 | 0.069 | 0.052 |

^{a}Mean of triplicates; p—power law parameter relating G′ and ω (G′ α ω^{p}); q—power law parameter relating G″ and ω (G″ α ω^{q}).
^{b}Sample did not show the viscoelastic behavior.
^{c}tWPC powder extruded with water only (non-pH-adjusted).

An important aspect of the oscillatory results is that pH adjustment accompanied with SC—CO₂ by SCFX process significantly altered the gel properties of WPC. One explanation is that when the net charge on the proteins was manipulated, there were significant changes in protein-protein, and protein-solvent interactions governed by shifting the balance of attractive and repulsive forces. This shift consequently affected the rate of aggregation, resulting in spatial arrangement of protein molecules and different gel structures depending on the extent of net charges (Clark, 1998; Tang, McCarthy, & Munro, 1995). When the SC—CO₂ was incorporated into the WP matrices, the porous structure of WP extrudates could be created by SC—CO₂ expansion at the die exit as the pressure quenched to atmospheric level. It is possible that this could cause the rapid and extensive hydration of tWPC powders even at ambient temperature. This is as would be expected if electrostatic forces are important since repulsion between like charges should lead to a more expanded and easily hydrated structure leading to stronger network as seen in some tWPC samples. Moreover, Zhong and Jin (2008) reported the improvement in gelling properties of WP upon heating after the WPC solution (10%, w/v) was treated with SC—CO₂ since the dissolved SC—CO₂ could alternate the secondary structure (α-helix, β-sheet, and random coil) of proteins (Liu, Hsieh, & Liu, 2004; Striolo, Favaro, Elvassore, Bertucco, & Di Noto, 2003). However, further studies on molecular and chemical changes of WPs induced by the SCFX process are needed to correlate with the rheological properties of tWPC products. From these findings, reactive SCFX process has been successfully integrated for texturization of WPC which exhibits cold-gelling behavior at ambient temperature.

### Temperature Stability of Selected tWPC

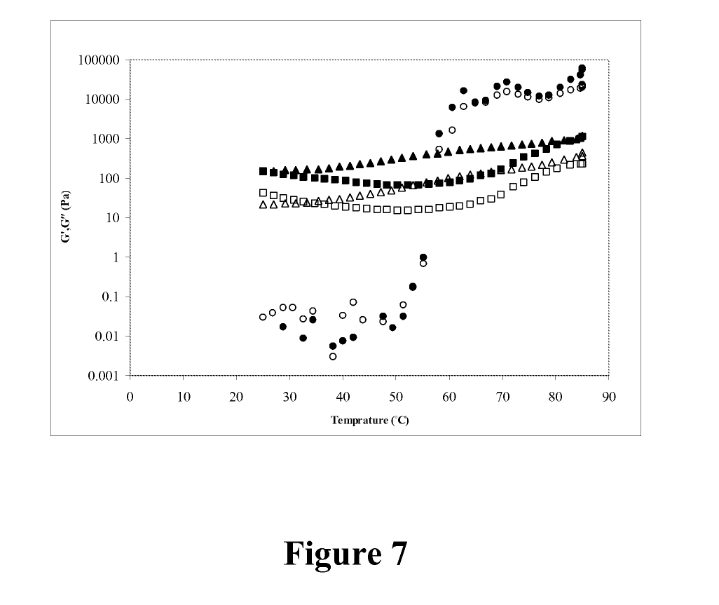

Based on the viscosity and viscoelastic behavior results, tWPC samples produced under extremely acidic (pH 2.89) and alkaline (pH 8.16) conditions with SC—CO₂ injection were selected as the best representatives for the thermal stability and further studies. FIG. 7 illustrates the variation of viscoelastic moduli (G′ and G″) of 20% (w/w) tWPC dispersions with temperature ramped from 25 to 85° C. The same heat treatment was done to 20% (w/w) unprocessed WPC dispersion. The hard and solid gel formation was found in the unprocessed WPC after the temperature was raised beyond the gelation temperature of native WPs to 85° C. On the other hand, both tWPC samples notably displayed little variation in G′ and G″ when the temperature was ramped from 25° C. to 85° C. The extreme acid-treated sample showed a gradual increase in G′ and G″ as temperature was increased from 25 to 85° C. This finding is in contrast to the results reported by Resch, Daubert, and Foegeding (2004) who found a decrease in viscosity of freeze-dried (12.2%, w/w) and spray-dried (10.2%, w/w) derivatized WPC dispersions as temperature was increased from 10 to 90° C. These derivatized WPs have been also reported to perform a cold-setting gel at ambient temperature.

In the case of extreme alkali-treated sample, G′ and G″ appeared unchanged as the temperature was elevated from 25 to 65° C., and slightly increased as temperature was raised to 85° C. A slight variation in G′ and G″ of both tWPC samples after the temperature ramping could be attributed to the additional unfolding and denaturation of native WPs remaining in tWPC powders. A noticeable difference between these two samples corresponding to the temperature ramp may be explained by the difference in the magnitude of WP aggregation in these samples. Our electrophoresis SDS-PAGE results showed the higher extent of large WP aggregates in alkali-treated sample. This could affect the amount of native WPs remaining in tWPC powders, resulting in slight difference in their temperature correspond. As expected, SCFX process rendered WPC into an ingredient having a stable cold-set gelling behavior over a wide range of temperature.

### Water Holding Capacity of Selected tWPC Powders

It was observed that alkali-treated tWPC extrudates had turbid, opaque, and slightly dark color appearance indicating large aggregates scattering light. It was reported that this normally occurred when WPs are denatured in the alkali condition (Onwulata et al., 2006). On the other hand, the acid-treated extrudates were slightly yellowish and transparent appearance. The similar observations in WPI extruded under acidic and alkaline conditions were also reported by Onwulata et al. (2006). They indicated the differences in structure of WPI aggregates produced under different pH conditions. The pH adjustment and ionic strength could affect the rates of unfolding, degree of denaturation, and aggregate size of WPs, resulting in different rheological characteristics and the quantification of water holding in final products (Alting, 2003). It is interesting to note that injecting SC—CO₂ into pH-adjusted WPC yielded a lighter product color, implying the porous structure through SC—CO₂ expansion in WP extrudates.

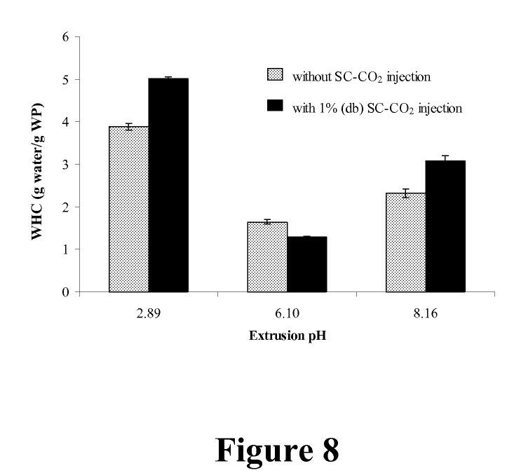

tWPC samples produced under extremely acidic (pH 2.89), alkaline (pH 8.16) conditions with and without SC—CO₂ injection compared with the non-pH adjusted sample (pH 6.10) were selected to study their water holding capacity (WHC). The comparison of WHC values among tWPC powders is demonstrated in FIG. 8. Without SC—CO₂ addition, the extreme acid-treated tWPC powder had highest WHC. This may be explained by the maximum solvent-protein interaction at extremely acidic condition (pH 2.89) due to unfolding of polypeptide chains which allowed exposure of more reactive amino acid side chains and thus, favoring water binding. At pH around 6.10 evident in non-pH adjusted tWPC, probably less protein-water interaction occurred because of the neutralized charges on amino acid side chains. Under alkaline condition, the insolubity of WPs increased due to the larger proportion of proteins being polymerized to higher molecular weight molecules and large aggregates, resulting in turbid and particulate type of gel (Onwulata et al., 2006). According to Bowland and Foegeding (1995), Elofsson et al. (1997), and Hudson et al., (2000), particulate gels are opaque and synerese which generally have lower water holding capacity due to the large inter-particle pores. On the other hand, WPs produced under acidic conditions (pH<3.5) were reported to form the translucent or fine-stranded type of gel with higher water holding capacity (Hudson et al., 2000; Ikeda & Morris, 2002). In addition, an increase in solubility and lower extent of aggregates of WPC produced under acidic condition was demonstrated by our protein solubility and SDS-PAGE results. This could be a reason of lower water absorption in alkali-treated tWPC powder compared to the acid-treated tWPC powder.

Addition of 1% (db) SC—CO₂ significantly enhanced the WHC of tWPC powders, except for the non-pH-adjusted sample. FIG. 8 clearly shows that the WHC of tWPC powders increased by 3 and 1.9 times for acid-treated and alkali-treated samples, respectively, compared to non pH-adjusted tWPC without SC—CO₂ addition. These results indicated the formation of porous structure through SC—CO₂ expansion in WP extrudates leading to the higher WHC and viscosity in tWPC samples as previously described.

### Conclusion

tWPC products demonstrated instant dispersibility and the ability to form a cold-setting gel without additional heat input. The 20% (w/w) tWPC dispersions exhibited shear thinning behavior, indicating a typical characteristic of thickening agents used in food system. Injection of 1% (db) SC—CO₂ to pH-adjusted protein polymer melts significantly enhanced the viscosity and other viscoelastic properties of most tWPC samples. Incorporation of SC—CO₂ to the extreme acid-treated tWPC showed the best rheological characteristics by contributing approximately 258 and 275,000 times higher apparent viscosity and elastic modulus than the unprocessed WPC. tWPC samples produced under extremely acidic (pH 2.89) and alkaline (pH 8.16) conditions with SC—CO₂ exhibited high stability of rheological properties over a wide temperature range (25 to 85° C.). Addition of SC—CO2 also increased the water holding capacity of pH-treated tWPC samples. The results confirm the hypothesis that reactive extrusion of WPs in highly alkaline or acidic environment combined with controlled shear and heat in the presence of mineral salts (CaCl₂ and NaCl) and SC—CO₂ favorably generated new WP ingredients with unique gelling and functional properties which may open up a new avenue for utilization of WP as a thickening or gelling agent in food formulations. Further studies are underway to understand the mechanisms of molecular and chemical changes of WPs induced by the SCFX process which could be correlated with the enhanced rheological properties of tWPC products.

### Example 4

### Emulsification Mechanisms and Characterizations of Cold, Gel-Like Emulsions Produced from Texturized Whey Protein Concentrate

Examples 4-6 correspond to various experiments performed to study emulsification mechanisms and characterizations of cold, gel-like emulsions produced from texturized whey protein concentrate.

A novel supercritical fluid extrusion (SCFX) process was used to successfully texturize whey protein concentrate (WPC) into a product with cold-setting gel characteristics that was stable over a wide range of temperature. It was further hypothesized that incorporation of texturized WPC (tWPC) within an aqueous phase could improve emulsion stability and enhance the rheological properties of cold, gel-like emulsions. The emulsifying activity and emulsion stability indices of tWPC and its ability to prevent coalescence of oil in water (o/w) emulsions were evaluated compared with the commercial WPC80. The cold, gel-like emulsions were prepared at different oil fractions (φ=0.20 to 0.80) by mixing oil with the 20% (w/w) tWPC dispersion at 25° C. and evaluated using a range of rheological techniques. Microscopic structure of cold, gel-like emulsions was also observed by Confocal Laser Scanning Microscope (CLSM). The results revealed that the tWPC showed excellent emulsifying properties compared to the commercial WPC in slowing down emulsion breaking mechanisms such as creaming and coalescence. Very stable with finely dispersed fat droplets, and homogeneous o/w gel-like emulsions could be produced. Steady shear viscosity and complex viscosity were well correlated using the generalized Cox-Merz rule. Emulsions with higher viscosity and elasticity were obtained by raising the oil fraction. Only 4% (w/w) tWPC was needed to emulsify 80% (w/w) oil with long-term storage stability. The emulsion products showed a higher thermal stability upon heating to 85° C. and could be used as an alternative to concentrated o/w emulsions and in food formulations containing heat-sensitive ingredients.

As shown in previous work, conformational structure and functionalities of whey protein concentrate (WPC) were modified through partial denaturation by means of combined treatments of highly acid treatment (pH<3.0) with heat, shear, and supercritical carbon dioxide (SC—CO₂) injection during a supercritical fluid extrusion process (SCFX) in the presence of optimum salt concentrations. Preliminary studies indicated that adding NaCl and CaCl₂ at concentration of ˜64 mM and 33 mM, respectively, to the WP blend could prevent excessive aggregation and promote a stronger structure of protein gels. The texturized WPC (tWPC) was found to possess extremely high viscosity with the ability to form a viscoelastic gel at ambient temperature, whereas the commercial WPC80 always produced a liquid dispersion due to its globular structure (Manoi & Rizvi, 2008).

The complex functions of tWPC such as high solubility, thickening properties, water holding capacity, and surface hydrophobicity have brought the innovative ideas of utilizing this WP derivative product as the polymeric surfactant or emulsifier/stabilizer for food emulsions. In this study, it was hypothesized that tWPC could enhance the stability of the emulsion by two major mechanisms; 1) an enhanced adsorption at the oil-water interface which could form a stronger, protective stabilizing layer, and 2) a rheology-modified continuous phase which could better maintain dispersed oil droplet. The improved the emulsifying properties of tWPC and its ability to prevent flocculation and coalescence of emulsions could open up new potential uses in food applications.

Prior to the present invention, cold, gel-like emulsions prepared with tWPC, the derivatized WP powder, at ambient temperature have not yet been reported. This approach could be beneficial for controlling the texture of emulsion-filled gel products and their derivatives. In this part of the study, it was hypothesized that incorporation of tWPC within an aqueous phase could enhance the rheological properties and thermal stability of o/w emulsions. The main objectives were to evaluate the emulsification properties of tWPC compared with the commercial WPC80 and to investigate the thermal stability and the effects of oil fractions (20 to 80%, w/w) on the rheological properties of gel-like emulsions.

### Example 5

### Materials and Methods

### Materials

A commercial WPC80 (lactalbumin-493) was obtained from Leprino Foods Company (Lemoore west, CA, USA). The compositions (dry basis) of the commercial WPC80 were 81.5% protein, 5.5% fat, 4% moisture, and less than 3% ash. Corn oil was purchased from a local retailer. Nile red and Fast Green FCF were obtained from Sigma-Aldrich (Sigma Chemical Co., St. Louis, Mo., USA). A powder blend comprising a mixture (by weight) of 94% prehydrated (10% wet basis) WPC80, 6% pre-gelatinized corn starch (Hammond, Ind., USA), 0.6% (WPC-starch basis) NaCl, and 0.6% (WPC-starch basis) CaCl₂ (Sigma Chemical Co., St. Louis, Mo., USA) were preconditioned at ambient temperature overnight before feeding to the extruder. In this process, the pre-gelatinized corn starch has been used as a binder to hold protein matrices because of their ability to form hydrogen bonds in the extruded product (Amaya-Llano, Morales Hernandez, Castaño Tostado, & Martinez-Bustos, 2007). Considerably, the pre-gelatinized corn starch acted as an inactive filler in the tWPC extrudate formation (Manoi & Rizvi, 2008).

### Production of tWPC by SCFX Process

A pilot-scale Wenger TX-52 Magnum (Wenger Manufacturing, Sabetha, Kans., USA) co-rotating twin screw extruder with a length to diameter ratio (L/D) of 28.5 was configured to operate at screw speed of 180 rpm and feed rate of 35 kg/h. The die was fitted with two circular inserts of 1.2 mm diameter each. The die pressure was maintained at 10-15 MPa for continuous SC—CO₂ flow into the protein polymer melt, at the desired rate (1% dry feed basis). The 15% (v/v) HCl solution stream was injected into the extruder at the mixing zone to create a pH of about 2.9 and the extrusion was conducted at 60% (dry feed basis) moisture content. The final product temperature was maintained at 90° C. at the die exit. The extrudate was collected, dried (5-7% moisture content), and grounded using a mill machine (Thomas-Wiley Mill model ED-5, Arthur H. Thomas Co., PA, USA) to reduce the particle size to less than or equal to 1 mm. The tWPC powder was then stored at room temperature in air tight containers until analyzed.

### Emulsifying Properties

### Emulsifying Activity Index and Emulsion Stability Index

The emulsifying activity index (EAI) and emulsion stability index (ESI) were determined by the turbidometric technique described by Pearce and Kinsella (1990) with some modifications. The emulsions were prepared from 10 mL corn oil and 40 mL of 3% (w/w) tWPC or commercial WPC80 dispersions, adjusted to pH 7. Sodium azide (0.04% w/w) was added to WP dispersions to prevent microbial growth. Emulsions were then mixed at 25° C. using a high-speed dispersing and emulsifying unit (model IKA-ULTRA-TURRAX® T25 basic, IKA® Works, Inc., Wilmington, N.C.) at 21,500 rpm for 2 min. Resulting o/w emulsions (10 μL) were then diluted in 5 mL of 0.1 M phosphate buffer containing 0.1% (w/v) sodium dodecyl sulfate (SDS). The absorbance of the diluted emulsions were then determined in a 1-cm path length cuvette at a wavelength of 500 nm in a Spectronic 1200 spectrophotometer (Bausch and Lomb, Rochester, N.Y., USA). The turbidity (T) of emulsions was calculated using the following formula:

$$T = \frac{2.303 xA}{l} \qquad ( 1 )$$

where A is the absorbance at 500 nm, and l is the path length of the cuvette (1 cm).

The emulsifying activity index (EAI) was then calculated as

$$EAI ( {m}^{2} {g}^{- 1} ) = \frac{2 xTxD}{φ xCx 10 , 000} \qquad ( 2 )$$

where T is the turbidity, D is the dilution factor, Φ is the volumetric fraction of oil, C is the weight of protein per unit volume of aqueous phase before the emulsion was formed (g mL⁻¹) and 10,000 is the correction factor for square meters. The EAI of emulsions was monitored after storage for 0, 1, and 24 h. The mean of three replicates is reported.

The emulsion stability index (ESI) was calculated after the emulsions were held at 4° C. for 24 h and reanalyzed for emulsion turbidity as described previously using the following formula:

$$ESI ( h ) = \frac{( Tx Δ t )}{Δ T} \qquad ( 3 )$$

where T is the turbidity value at 0 h, ΔT is the change in turbidity during the storage period, and Δt is the time interval. The mean of three replicates is reported.

### Creaming Index

Dispersions containing various concentrations of tWPC or commercial WPC80 (0.25, 0.5, 1, 2, 3, and 4%, w/w) in 0.1 M phosphate buffer at pH 7 were prepared at ambient temperature. Sodium azide (0.04% w/w) was added to WP dispersions to prevent microbial growth. Ten millimeters (10 mL) of corn oil and 40 mL of WP dispersion were then mixed at 25° C. using a high-speed dispersing and emulsifying unit (model IKA-ULTRA-TURRAX® T25 basic, IKA® Works, Inc., Wilmington, N.C.) at 21,500 rpm for 2 min. The creaming index was evaluated as described by Firebaugh and Daubert (2005) with some modifications. Ten millimeters (10 mL) of each emulsion was filled into a glass test tube (1.5-cm internal diameter×12-cm height) and then stored at ambient temperature. The height of the serum (Hₛ) and the total height of emulsions (Hₜ) were recorded after storage at ambient temperature for 1, 7, and 14 days. The mean of three replicates is reported. The creaming index was reported as:

$$Creaming Index ( \% ) = \frac{{H}_{s}}{{H}_{t}} x100 \qquad ( 4 )$$

### Cold, Gel-Like Emulsion Preparation

Dispersions containing 20% (w/w) tWPC or commercial WPC80 were prepared in deionized water and stirred for at least 2 h at ambient temperature, and then stored overnight at 4° C. to ensure complete dissolution. Sodium azide (0.04%, w/w) was added to WP dispersions to prevent microbial growth. Emulsions containing oil levels of 20 to 80% (w/w) (φ=0.20, 0.30, 0.40, 0.50, 0.60, 0.70, and 0.80) were prepared for studying the effect of oil concentrations on emulsion properties. Thus, in this study the protein concentration in aqueous phase was fixed at 20% (w/w). The emulsion of a given oil concentration was prepared by mixing the correct amount of corn oil with the appropriate quantity of aqueous tWPC dispersion, at 9,500 rpm for 3 min using a high-speed dispersing and emulsifying unit (IKA-ULTRA-TURRAX® T25 basic, IKA® Works, Inc., NC, USA). In case of emulsions containing ≧60% oil, the pre-emulsion containing 50% oil was first prepared as above. The appropriate amount of oil was then added to the pre-emulsion at the rate of 10 mL/min. The emulsion was continuously mixed while oil was added using a Sunbeam Mixmaster beater at speed 5 until the final emulsion was obtained. The resulting emulsions were then stored in sealed containers at ambient temperature until analyzed.

### Rheological Characterization of Cold, Gel-Like Emulsions

The rheological properties of cold, gel-like emulsions were evaluated using a strain-controlled rheometer (ARES, TA Instruments, New Castle, Del., USA) equipped with a Peltier temperature controlling system. A cone and plate geometry (diameter=25 mm, nominal con angle=0.1 radians) was used for steady shear viscosity measurements and the parallel plate geometry (diameter=25 mm, sample thickness=2 mm) was used for small-amplitude oscillatory shear experiments. A thin layer of mineral oil was applied to the exposed sample edges to prevent the moisture loss. All measurements were conducted at 25° C.

### Steady Shear Viscosities

Shear rate was ramped from 1 to 100 s⁻¹. Shear stress, shear rate, and steady shear (apparent) viscosity (η) were recorded by TA Orchestrator software.

### Viscoelastic Properties by Small-Amplitude Oscillatory Shear (SAOS)

The frequency was oscillated from 0.1 to 100 rad/s and all measurements were performed within the identified linear viscoelastic region and made at 1% strain. The elastic modulus (G′), loss modulus (G″), complex viscosity (η*), and loss tangent (tan δ) were then recorded by TA Orchestrator software.

### Thermal Stability

The temperature was raised from 5° C. to 85° C. at 2° C./min heating rate and at a constant frequency rate of 1 rad/s and 1% strain. The elastic modulus (G′) was then recorded by TA Orchestrator software.

### Confocal Microscopy

The selected emulsion samples were stained with a mixture of Nile Red (0.01%, w/w in a mixture of polyethylene glycol, glycerol, and deionized water (50/45/5)) to visualize the oil phase and Fast Green FCF (0.001%, w/w in deionized water) to visualize the protein phase. The stained emulsion was placed on a glass slide and covered with a cover slide. Confocal laser scanning microscopy (CLSM) was performed on a Leica TCS-SP2 Confocal Laser Scanning head mounted on a Leica DMRE-7 (SDK) upright microscope (Leica Microsystems Inc., Bannockburn, Ill., USA) equipped with a 20×HC PL APO/0.70NA oil immersion objective lens. Confocal illumination was provided by an Argon laser with excitation at 488 nm and a Helium Neon laser (HeNe) with excitation at 633 nm. The green emission range was 500-580 nm and red emission range was 650-730 nm.

### Statistical Analysis

Statistical analysis was done using MINITAB® release 15 statistical software (State College, Pa., USA). Significant differences (p<0.05) were determined by analysis of variance using the general linear models and least square means procedure.

### Example 6

### Results and Discussion

### Emulsifying Properties

The emulsifying activity index (EAI) is related to the surface area stabilized by a unit weight of proteins. It represents the ability of proteins to be adsorbed at the interface of fat globules and the aqueous phase (Pearce & Kinsella, 1978). The results shown in Table 4 reveal that the EAI of commercial WPC80 and tWPC was not significantly different (p<0.05) at zero-time after emulsion preparation. Graham and Phillips (1980) described that the commercial WPC80 which is mainly composed of native proteins, a highly structured molecule, could be adsorbed at the interface by forming a strong viscoelastic film around the fat globules. On the other hand, the tWPC composed of partially denatured and aggregated proteins as indicated in our previous works possibly achieves the emulsion formation activity by different approach. It is possible that structural changes in tWPC due to denaturation and polymerization induced by reactive SCFX process lead to an increased surface hydrophobicity and molecular flexibility, allowing an effective adsorption of protein molecules at the oil-water interface. It is well documented that denatured proteins usually exhibit a high surface hydrophobicity which enhances emulsifying activity and interfacial concentration by contributing to the film rigidity through hydrophobic interactions between adjacent protein molecules at the interface (Guilmineau & Kulozik, 2007; Kato & Nakai, 1980; Matsudomi, Sasaki, Kato, & Kobayashi, 1985; Mitidieri & Wagner, 2002).

TABLE 4
Emulsifying properties of 3% (ww) WP

| | Emulsion activity index (EAI) | | |
|---|---|---|---|
| | | | Emulsion |
| | $EAI ( {m}^{2} {g}^{- 1} ) = \frac{2 xtxD}{φxCx 10 \text{,} 000}$ | | stability index (ESI) |
| Sample | 1 hr storage | 24 hr storage | $ESI ( hr ) = \frac{( Tx Δt )}{Δ T}$ |
| Commercial | 362.08 ± 0.34 | 112.85 ± 0.44 | 32.70 ± 0.08 |
| WPC80 | | | |
| Texturized | 431.94 ± 0.78 | 431.68 ± 0.77 | 13.504 ± 13.85 |
| WPC80 | | | |

Interestingly, over longer periods of storage of 1 to 24 h, the EAI of commercial WPC80 was observed to decrease, whereas that of tWPC remained unchanged. The decrease of EAI as a function of time reflects the instability of the commercial WPC80-stabilized emulsion. According to Dalgleish (1997), instability of emulsions occurs when there is either insufficient surfactant to cover the entire oil-water interface or there are gaps in the interfacial layers, thereby decreasing the total adsorbed surface. The emulsion stability index (ESI), on the other hand, reflects the ability of proteins to impart strength to emulsion for resistance against coalescence upon storage (Patel & Kilara, 1990). The greater ESI was observed for tWPC (ESI=13,504 h) compared to that of commercial WPC80 (ESI=33 h) (Table 4), indicating that the emulsion stabilized by tWPC was remarkably more resistant to coalescence. These results imply that the emulsion stability and surface behaviors of native proteins are limited possibly due to their rigid packed globular conformation along with their less molecular flexibility (Kinsella, 1979; Wagner & Guéguen, 1999).

According to the results, the high stability upon storage of diluted emulsions prepared with tWPC is likely due to the formation of a rigid film preventing coalescence of the droplets. This is similar to those reported for some water soluble ampiphilic polymers or macromolecular emulsifiers (Akiyama, Kashimoto, Fukuda, Hotta, Suzuki, & Kitsuki, 2005; Akiyama, Yamamoto, Yago, Hotta, Ihara, & Kitsuki, 2007; Sun, Sun, Wei, Liu, & Zhang, 2007). It is important to note that an essential function of surfactants or emulsifiers is not only that they produce the equilibrium interfacial tension, but also they impart rigidity to the interface by forming films which provide strong repulsive forces between droplets due to a combination of electrostatic and steric interactions, and resistance to rupture (Lucassen-Reynders, 1993; McClements, 1999b). Moreover, viscosity and gelling behaviors of tWPC possibly reflect the hydrodynamic properties of protein macromolecules, in particular, their shape and size. It has been reported that emulsion flocculation and coalescence could be prevented by inducing a heavily hydrated, charged and a thick interfacial layer (Dagorn-Scaviner, Guéguen, Lefebre, 1987; Graham & Phillips, 1976). These studies also emphasized the importance of the thickness and charge of the protein interfacial layer in preventing coalescence of emulsions. Based on the observations noted above, it is reasonable to speculate that the higher surface activity and better emulsion stability of tWPC compared to the commercial WPC80 could be based on the formation of a thick, rigid film at the oil-water interface through hydrophobic interactions between protein molecules at the interface, and an increase in viscosity of the continuous phase of emulsion.

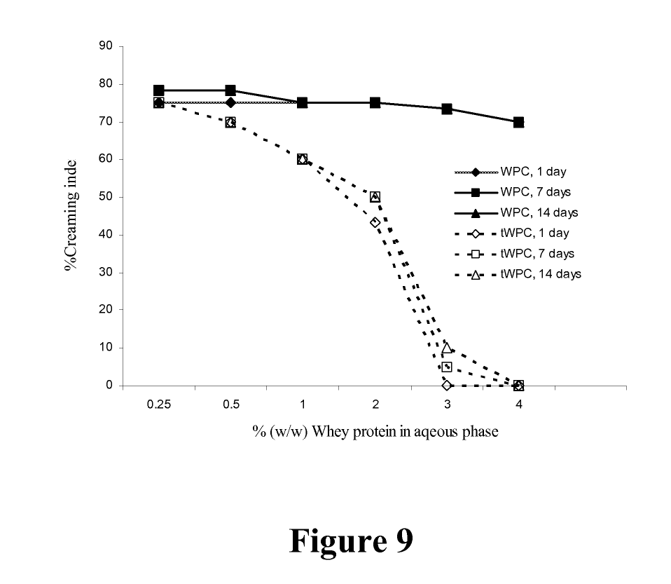

Emulsion stability could be also observed with respect to creaming and coalescence. In general, coalescence can induce oiling-off and accelerate creaming, which in turn reduces the shelf life of o/w emulsions. The creaming index has been used to indicate the susceptibility of oil droplets to coalescence by such forces as gravitational, colloidal, hydrodynamic, and mechanical, and the resistance of the droplet membrane to rupture during a certain period of time (McClements, 1999b; Pearce & Kilara, 1978). In this study, the effect of protein concentration on the creaming index was also monitored. As depicted in FIG. 9, the emulsions stabilized by commercial WPC80 showed separation starting from day 1 of storage following homogenization, whereas the creaming index of tWPC was time and protein concentration dependent. Clearly, the emulsion stability was enhanced by increasing protein concentration. For instance, at lowest protein concentration of 0.25% (w/w), the tWPC-stabilized emulsion was most susceptible to creaming, while the highest protein concentration of 4% was the least susceptible (FIG. 9). Upon dilution, such emulsions tend to lose the protein stabilization effect. However, at equal protein concentrations, emulsions stabilized by commercial WPC80 coalesced more rapidly than those made with tWPC. The tWPC exhibited lower creaming index at higher protein concentrations. At 2% (w/w) protein concentration, the commercial WPC80 showed nearly twice as much creaming as tWPC. It should be noted that emulsions stabilized with 3% (w/w) tWPC showed little phase separation (creaming index=7%) even after 14 days of storage at 25° C., no phase separation was observed in emulsions containing higher protein concentrations (≧4%, w/w).

Similar results were obtained in emulsions stabilized by derivatized WPs (Firebaugh & Daubert, 2005), protein aggregates (Rosa et al., 2006), and polymeric surfactants (Akiyama et al., 2005, 2007; Sun et al., 2007). Patel and Kilara (1990) and Yamauchi, Shimizu, and Kamiya (1980) indicated that a positive correlation between protein content and emulsion stability was attributed to an increase in the viscosity of the continuous water phase and the amount of protein adsorbed at the fat globule surface. The viscosity of continuous phase is important in predicting the creaming rate of emulsions. According to the Stokes equation (Eq. (5)), the rate of phase separation (ν) between the continuous phase and dispersed phase depends on densities of the two phases (ρ₁ and ρ₂), the gravity (g), the radius of particles (r), and the viscosity (η) of the continuous phase (Roland, Piel, Delattre, & Evrard, 2003).

$$v = \frac{2 {r}^{2} ( {ρ}_{1} - {ρ}_{2} ) g}{9 η} \qquad ( 5 )$$

As shown in the experimentals provided herein above, the tWPC was found to possess remarkably higher viscosity compared to the commercial WPC80 at equal protein concentrations (Manoi & Rizvi, 2008). Furthermore, the thickness of the adsorbed protein layer could be another significant factor in the stability of emulsions. Rosa et al. (2006) reported that the thickness of adsorbed aggregated protein layer on the droplet surface is higher than that of a native protein by approximately 20 to 26 times.

### Rheological Characterization of Cold, Gel-Like Emulsions

### Steady Shear Viscosities

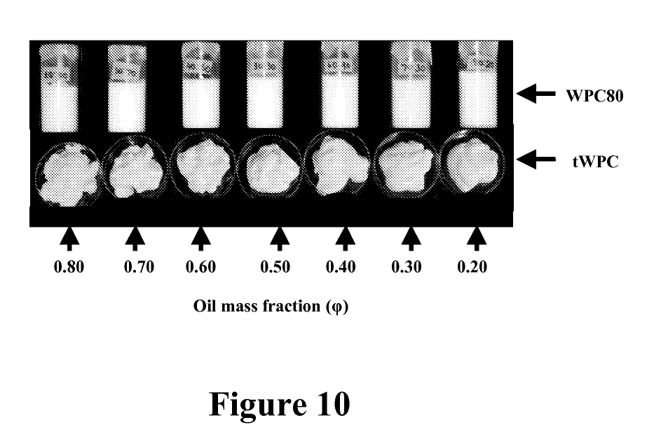

As shown in the experimentals provided herein above, it was revealed that the tWPC formed a cold-set thickening ability upon reconstitution with water at 20% (w/w) protein concentration (Manoi & Rizvi, 2008). In this study, it was expected that both an associative thickening of tWPC and a protective stabilizing layer on oil droplets would yield stable gel-like emulsions prepared at ambient temperature. Once the oil concentrations in the emulsion system decreased from 80 to 20% (w/w), the final protein and water contents of emulsions would vary from 4 to 16% (w/w), and 16 to 64% (w/w), respectively. Visual observations of emulsions after one day of storage at 25° C. revealed that all emulsions made from commercial WPC80 remained liquid, while those stabilized by tWPC displayed a self-standing gel with a soft solid-like texture as shown in FIG. 10.

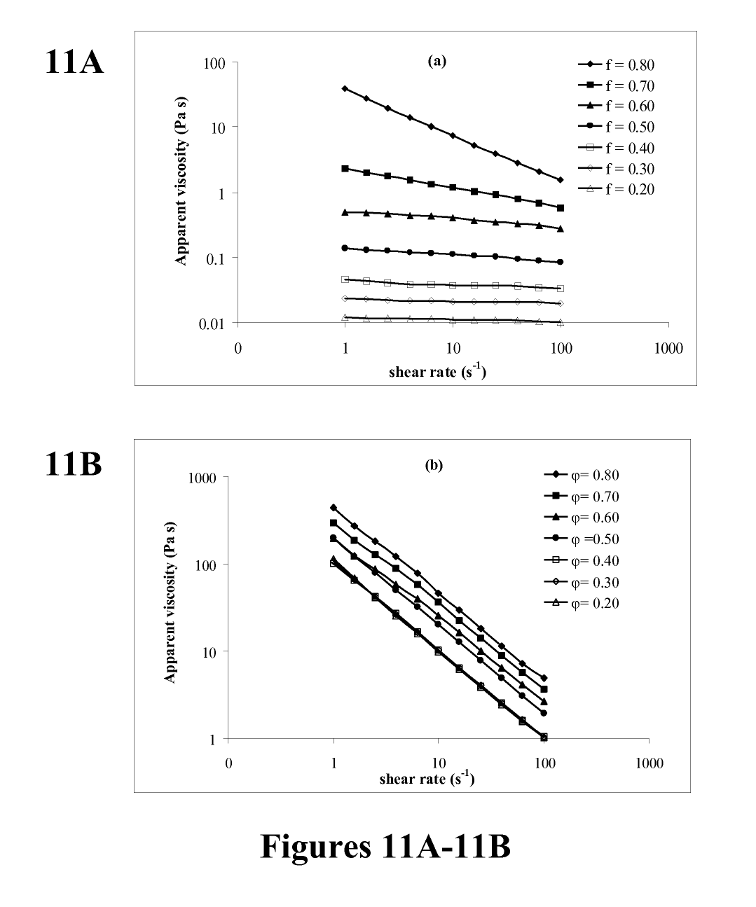

Changes in viscosities as a function of shear rates of emulsions stabilized by commercial WPC80 and tWPC at various oil mass fractions (φ=0.20-0.80) are presented in FIGS. 11A and 11B, respectively. In general, it showed that at higher oil fractions, emulsions exhibited higher viscosity at a given shear rate. Emulsions prepared with commercial WPC80 exhibited almost Newtonian behavior at oil fractions of 0.20 to 0.60. FIG. 11A clearly shows the relative viscosity of such emulsions did not change with shear rate. Dimitrova and Leal-Calderon (2004) described that non-flocculated samples generally exhibit a Newtonian flow, while the shear thinning behavior of emulsion is associated with the flocculation of fat droplets. Similar observations were also discussed by Boutin et al. (2007), and Demetriades, Coupland, and McClements (1997). In addition, it was observed that reducing the proportion of oil in commercial WPC80-stabilized emulsions created higher phase separation rate. This is because the interactions between droplets were weakened and emulsions became less stable (Depree & Savage, 2001). However, the commercial WPC80-stabilized emulsions at higher oil fractions of 0.70 and 0.80 exhibited shear-dependent fluids with shear-thinning behavior indicated by the decrease of apparent viscosity with shear rate (FIG. 11A). It was elucidated that in concentrated emulsions, the droplets were close enough to interact with each other and form the network of aggregated droplets which deformed and consequently disrupted when applied shear rate was increased, resulting in viscosity reduction (Ma & Barbosa-Cánovas, 1995; McClements, 1999a; Liu, Xu, & Guo, 2007). This, they suggested, gives emulsions the higher viscosity due to the flocculation of adjacent oil droplets to form a network. At higher oil concentration, the larger contact surface area between oil droplets opposed the free flow of the emulsion in a shear field, hence increasing its viscosity.

It is interesting to note that when the oil fraction was concentrated up to or above the close packing attainable in a dispersion of monodispse particles (φ*=0.64), the formation of a flocculated emulsion network and retarded coalescence of emulsions was strongly expected (Campanella, Dorward, & Singh, 1995; Dimitrova & Leal-Calderon, 2004; Turgeon, Sanchez, Gauthier, & Paquin, 1996). The authors also mentioned that since the droplets are fluid, they are susceptible to deformation. It means that emulsions can be concentrated up to volume fractions much higher than φ*. Similar explanations for the rhelogical behavior of highly concentrated emulsions such as mayonnaise which generally contains about 70-80% (w/w) oil have been pointed out by Ma and Barbosa-Cánovas (1995). Depree and Savage (2001) stated that the oil may account for 75% or more of the total volume of mayonnaise in which the oil droplets become distorted from their normal, spherical shape, and gives the traditional mayonnaise its high viscosity. However, our emulsion system was also probably close to that for polydisperse spheres. Therefore there may not be enough room for the droplets to move past each other, and so hydrodynamic effects may be as important as the viscosity in controlling stability i.e. it may be close to or above the so-called jamming transition. Bécu, Manneville, and Colin (2006) stated that the effect of inter-particle forces was convincingly demonstrated with moderately concentrated emulsions (φ=0.73), for which jamming transition was observed in the systems with attractive inter-droplet forces only.

Emulsions based on tWPC at all oil concentrations, on the other hand, were very stable upon storage. Apart from its excellent emulsifying properties, tWPC behaved as a gelling agent which could increase the viscosity of the continuous phase. The foregoing results indicated that the apparent viscosity of the 20% (w/w) tWPC dispersion (η_{6.31}=7.46 Pa s) was approximately 827 times higher than that of the commercial WPC80 (η_{6.31}=0.009 Pa s). Thus, the viscosity enhancement of continuous phase is one of the major keys in controlling rheological properties of prepared emulsions. It is believed that the influence of relatively higher viscosity, caused by tWPC in the aqueous phase, resulted in remarkably high emulsion stability even at lower oil contents.

A noticeable shear thinning behavior of emulsions in the presence of tWPC was observed at all oil fractions (FIG. 11B). The apparent viscosities at the same shear rate of 6.31 s⁻¹ (η_{6.31}) of emulsions based on the commercial WPC80 and tWPC at various oil fractions are compared in Table 5. It shows that the apparent viscosities of emulsions with tWPC were significantly (p<0.05) higher than those of emulsions with commercial WPC80 at comparable oil concentration. In addition, a greater apparent viscosity was also generally observed for emulsions containing higher oil fraction at a given shear rate as shown in FIG. 11A, FIG. 11B, and Table 5. However, the oil fraction was shown to have less influence on the viscosity for emulsions stabilized by tWPC than in the commercial WPC80. As indicated in Table 5, the apparent viscosity (η_{6.31}) increased by approximately 5 and 1000 times for emulsions stabilized by tWPC and commercial WPC80, respectively, as the oil fraction increased from 0.20 to 0.80. A slight change in viscosity of emulsions based on tWPC with oil fraction may be caused by a counterbalance of the reasonably high viscosity of the continuous phase of emulsions. It was expected that the higher protein content that remained in an aqueous phase participated in the properties of the lamella between the oil droplets, leading to the viscosity enhancement of emulsions.

TABLE 5
Rheological parameters^{a} of emulsions stabilized with commercial WPC80
and tWPC at various oil mass fractions

| Samples/ | | | | | |
|---|---|---|---|---|---|
| Oil mass | η_{6.31}^{b} | Mechanical spectra^{c} | | | |
| fractions (φ) | (Pa s) | G′ (Pa) | G″ (Pa) | η* (Pa s) | tan δ |
| Commercial | | | | | |
| WPC80 | | | | | |
| 0.80 | 9.95^{b} | 27.90^{a} | 5.37^{a} | 28.41^{a} | 0.19^{b} |
| 0.70 | 1.32^{a} | 5.60^{a} | 3.69^{a} | 6.62^{a} | 0.68^{a} |
| 0.60 | 0.42^{a} | —^{d} | — | — | — |
| 0.50 | 0.11^{a} | — | — | — | — |
| 0.40 | 0.04^{a} | — | — | — | — |
| 0.30 | 0.02^{a} | — | — | — | — |
| 0.20 | 0.01^{a} | — | — | — | — |
| tWPC | | | | | |
| 0.80 | 77.78^{g} | 1301.60^{f} | 90.79^{f} | 1304.75^{f} | 0.069^{f} |
| 0.70 | 57.36^{f} | 1001.51^{e} | 70.37^{d} | 1003.98^{e} | 0.070^{e} |
| 0.60 | 39.70^{e} | 894.88^{d} | 77.16^{e} | 898.19^{d} | 0.086^{d} |
| 0.50 | 31.83^{d} | 876.34^{d} | 75.07^{e} | 879.54^{d} | 0.086^{d} |
| 0.40 | 16.65^{c} | 468.29^{bc} | 50.31^{c} | 470.88^{c} | 0.107^{c} |
| 0.30 | 16.37^{c} | 491.59^{c} | 48.45^{c} | 493.97^{c} | 0.099^{c} |
| 0.20 | 15.80^{c} | 415.32^{b} | 40.47^{b} | 417.28^{b} | 0.097^{c} |

^{a}Values are the means of three replications, in each column different superscript letters denote significant differences between samples (p < 0.05).
^{b}The apparent viscosity was compared at shear rate of 6.31 s⁻¹.
^{c}The mechanical spectra were compared at 1 rad/s.
^{d}Emulsions were too liquid and unstable to allow dynamic testing.

Results revealed that only 4% (w/w) tWPC (in the final emulsion) was needed to emulsify 80% (w/w) oil. Despite the high oil content relative to water, it was observed that matrix was still o/w emulsion. This indicates the oil droplet entrapment ability, and reduced mobility of droplets and collision frequency of tWPC. Several studies have indicated that the viscosity of the continuous phase of the emulsions and the absorption of the polymers at the oil-water interface are the important keys for the stabilization of the o/w emulsions (Dickinson, 1995; Sun et al., 2007). McClements (2000) reported the thickening agents such as polysaccharides are usually added to o/w food emulsions to enhance the viscosity of the aqueous phase. At sufficiently high concentrations, creaming is retarded because the droplets are incapable of moving in the high viscosity or the gel-network formed by polysaccharides. Nevertheless tWPC was shown to be more advantageous than polysaccharides as an emulsifier by forming an adsorbed layer at the oil-water interface to form a protective steric barrier around droplets, while polysaccharides are usually identified as non-absorbing colloidal particles (Dalgleish, 2006; McClements, 2000). However, emulsification behaviors of tWPC are believed to be more similar to some polysaccharide derivatives, especially the hydrophobically modified water-soluble polymers based on an associative thickening characteristics and the adsorption of polymers at the interface (Akiyama et al., 2005, 2007; Sun et al., 2007).

### Viscoelastic Properties

The oscillation test was performed to establish the relationships between internal structure and flow of emulsions since the viscosity measurement alone was not capable of giving a better understanding of the link between the structure and macroscopic measurable properties. The small strain measurement is believed to leave the microstructure intact and thus makes it possible to characterize the viscoelastic properties of the original sample structure. Emulsions made with commercial WPC80 at oil fractions of 0.20 to 0.60 were not included in viscoelastic results because their very poor consistency prohibited measurement of their dynamic properties in the linear viscoelastic range. In most cases, the loss modulus (G″) was higher than the elastic modulus (G′) for all frequencies studied, which is a typical behavior of non flocculated or weakly flocculated emulsions, making it impossible to obtain the plateau modulus (Raymundo, Franco, Empis, & Sousa, 2002). Emulsions made with commercial WPC80 at oil fractions of 0.70 and 0.80 exhibited relative viscous characteristics of a viscoelastic liquid-like material observed from their high frequency-dependence of dynamic moduli. For comparison purpose, G′, G″, η*, and tan δ at frequency of 1 rad/s of emulsions based on commercial WPC80 and tWPC were summarized in Table 5. Notably, emulsions stabilized by tWPC investigated here, significantly (p<0.05) yielded higher G′, G″, and η* than those of commercial WPC80 at comparable oil concentrations. The elasticity of emulsion gels with tWPC was approximately 178 and 46 times higher than those of emulsions with commercial WPC80 at oil fraction of 0.70 and 0.80, respectively.

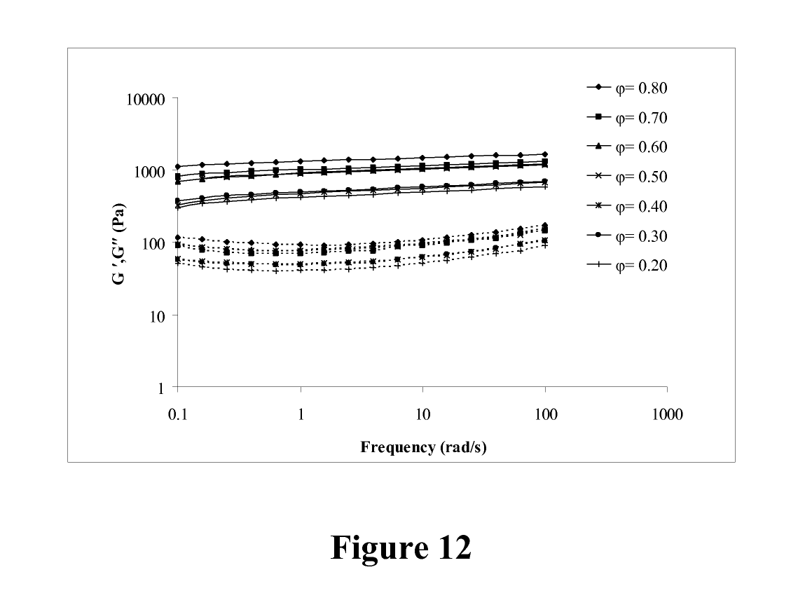

Results for tWPC stabilized emulsions showed that dynamic moduli were essentially frequency independent, over the frequency range considered (FIG. 12). The G′ values were approximately one order of magnitude higher than G″ values (FIG. 12 and Table 5). It could be interpreted that all emulsion samples behaved in a solid-gel like manner (Clark & Ross-Murphy, 1987). To confirm their gel behaviors, in most of the emulsions studied, a plateau region in the frequency range studied was found. The elastic modulus G′ tended to level off when the frequency was very low and close to zero (FIG. 12), implying that the strong interactions mainly contributing to the elastic modulus needed a long time to relax. At low frequency (long oscillation times), gel-type materials still possessed permanent interactions which gave a predominantly solid behavior. Dickinson and Hong (1995) explained that the development of an entanglement network between adsorbed and non-adsorbed protein molecules is mainly responsible for high elastic modulus and gel-like structure.

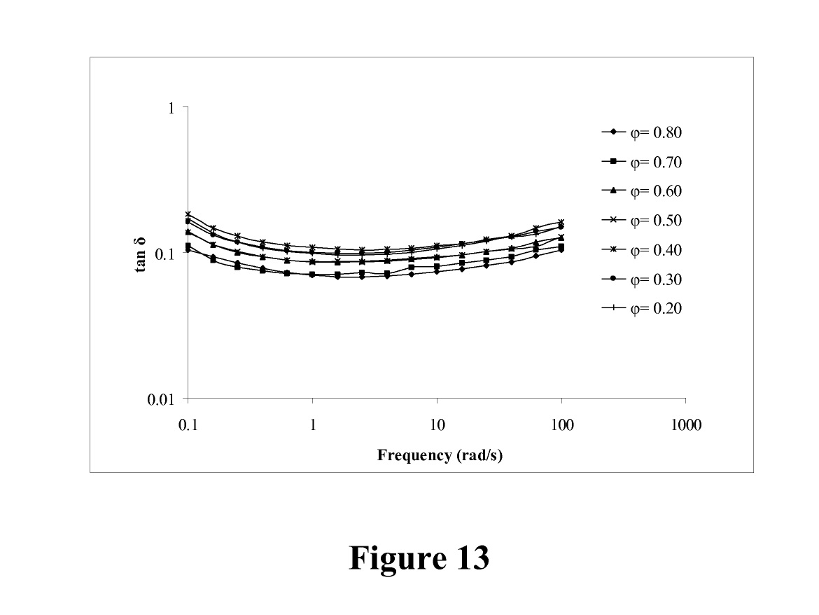

Loss factor, tan δ (G″/G′), compares the amount of energy lost to the amount of energy stored indicating whether elastic or viscous properties predominate in a sample. Results in Table 5 indicate that the commercial WPC80-stabilized emulsion at oil fraction of 0.80 had tan δ of 0.19 representing the characteristics of weak predominantly viscous gels, while the emulsion based on oil fraction of 0.70 was rather liquid (tan δ=0.68). On the other hand, emulsions based on tWPC had tan δ in a range of 0.07 (φ=0.80) to 0.10 (φ=0.20), expressing predominantly strong elastic gels. The variation of tan δ values with a range of frequencies for emulsions stabilized by tWPC at different oil fractions is presented in FIG. 13. The slope of tan δ for most cases was near zero (flat) indicating the frequency independency of the tan δ which also supported the relatively high elasticity of emulsion gels.

The dynamic mechanical moduli of emulsions showed oil concentration dependence. At higher oil fractions, the noticeably larger G′ and G″ of both commercial WPC80 and tWPC-stabilized emulsions were shown over an entire range of frequency (FIG. 12 and Table 5). It was also observed that emulsions made with higher oil fractions gave slightly lower tan δ. The increase in G′ with oil concentration indicates a more solid, gel-like structure of emulsions. Similar observations have been reported in mayonnaise with xanthan gum (Ma & Barbosa-Cánovas, 1995), highly concentrated protein-stabilized emulsions (Dimitrova & Leal-Calderon, 2004; Hemar & Horne, 2000; Raymundo et al, 2002), heat-set WP-stabilized emulsion gels (Chen & Dickinson, 1998), and cold-set WP-stabilized emulsion gels (Boutin et al., 2007; Rosa et al., 2006; Sok Line et al., 2005). They concluded that at higher oil concentrations, emulsions had more pronounced gel-like characteristics due to more packing and larger size of oil droplets in higher oil concentrations than in the lower oil concentrations.

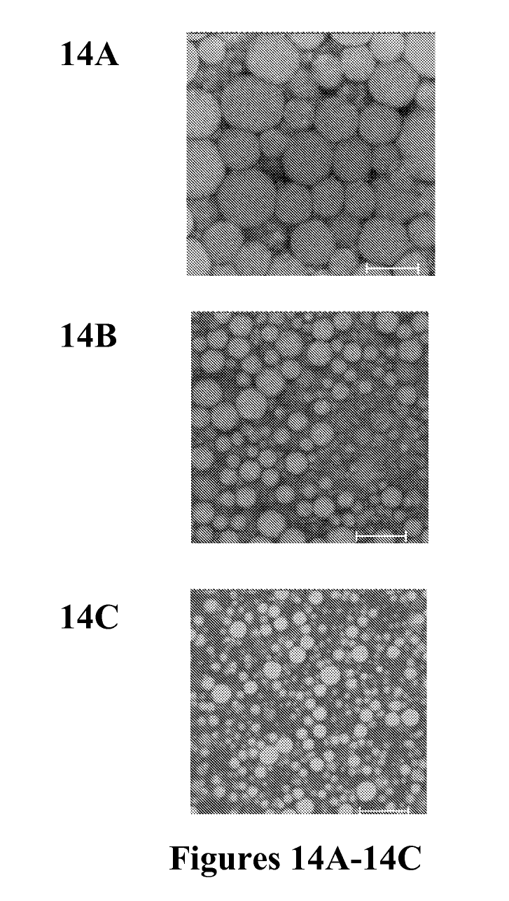

Microstructure analysis was carried out to determine the distribution of oil droplets in the WP matrix. FIGS. 14A-14C illustrate that oil droplets were homogeneously distributed in the continuous phase of WP gels. These CLSM observations strongly suggest that the fat globule size was larger and closely packed when the oil fraction was higher. The rheological results are well supported by CLSM images observed in selected tWPC stabilized emulsions at three different oil fractions of 0.80, 0.50, and 0.20. The samples that had more compact structure had the higher elastic modulus and lower tan δ. Another possible explanation for the corresponding increase in the elasticity of concentrated emulsions could be droplet repulsion and deformation. As discussed by Dimitrova and Leal-Calderon (2004) above the close packing of equivalent suspension of monodisperse spheres, φ*=0.64, the adjacent droplets forced together will begin to deform before their interface will actually touch due to the repulsive interactions between the droplets, and the emulsions become remarkably rigid and resemble an elastic solid.

Moreover, Chen and Dickinson (1998) pointed out that the dispersed oil droplets can help to build up the gel matrix structure and significantly enhance the gel strength of protein-coated emulsions when they act as the active filler. Many investigations have reported that the oil droplets acted as active fillers, for instance, in heat-set WP-stabilized emulsion gels (Chen & Dickinson, 1998, 1999; Dickinson & Hong, 1995, 1997; Jost et al., 1986; Matsumura, Kang, Sakamoto, Motoki, & Mori, 1993; Yost & Kinsella, 1992), and heat-set modified soy protein isolate-stabilized emulsion gels (Puppo, Sorgentini, & Añón, 2003) due to an increased gel strength with higher oil volume fractions. Based on dynamic mechanical properties of emulsions stabilized by tWPC investigated here, the oil droplets considerably behaved as active fillers and reinforced the gel strength through interactions between adsorbed proteins and those in the gel matrix.

### Bohlin's Parameters

The Bohlin's parameters relating G′ to frequency were calculated according to the cooperative theory of flow explained by Bohlin (1980) using the following power law model:

G′=Aω^{l/z} (6)

where G′ is the elastic modulus (Pa), ω is the frequency (rad/s), and z (dimensionless) and A (Pa) are the power law parameters. According to Bohlin's theory, z is a measure of the extent of the three dimensional network representing the level of these interactions and the coefficient A represents the order of magnitude of the interaction (Peressini & Sensidoni, 2000; Peressini, Sensidoni, & de Cindio, 1998). The values of z and A for all emulsions are summarized in Table 6. The z values ranged from 11.16 to 18.45 for emulsions based on tWPC with 20 to 80% (w/w) oil concentration (φ=0.20-0.80). From the results, emulsions prepared with 80% (w/w) oil concentration (φ=0.80) yielded the highest z value and coefficient A suggesting the most complex structure and the highest level of interactions between protein-coated droplets. It can be asserted that the compact packing of oil droplets in the protein network is responsible for elastic properties and deformation resistance of the emulsions. According to Peressini et al (1998), the coordination degree between rheological units (z) and on interaction strength (A) can be correlated with emulsion stability. The authors suggested that low values of z and A meant the tendency of oil droplets to coalesce when the emulsion undergoes mechanical stress. The results indicated the significantly high stability of all emulsions stabilized by tWPC. It was observed that all emulsions based on tWPC were soft solid-like in texture and very stable with long-term storage (≧6 months). In contrast, the oil release was visible in the majority of emulsions stabilized by commercial WPC80 after a short-time of storage. These findings are clearly related to emulsification activities of both proteins and their ability to stabilize emulsions as discussed in the first part of this work.

TABLE 6
Power law parameters^{a} (A and z) of emulsions
stabilized with commercial WPC80 and
tWPC at various oil mass fractions

| Samples/Oil mass | Power law parameters | | |
|---|---|---|---|
| fractions (φ) | A (Pa) | z | R² |
| Commercial WPC^{b} | | | |
| 0.80 | 28.08 | 6.52 | 0.97 |
| 0.70 | 5.33 | 2.61 | 0.99 |
| tWPC | | | |
| 0.80 | 1286.40 | 18.45 | 0.99 |
| 0.70 | 982.53 | 16.29 | 0.98 |
| 0.60 | 872.82 | 13.79 | 0.97 |
| 0.50 | 857.02 | 14.10 | 0.98 |
| 0.40 | 451.49 | 11.16 | 0.96 |
| 0.30 | 478.85 | 12.22 | 0.98 |
| 0.20 | 401.56 | 11.78 | 0.97 |

^{a}Values are the means of three replications; A—the proportional coefficient; z—the coordination number (G′ = A ω^{1/z}).
^{b}Emulsions at φ = 0.20 to 0.60 were too liquid and unstable to allow dynamic testing.

### Applicability of Cox-Merz Rule Between Steady Shear and Oscillation

The empirical Cox-Merz rule states that the magnitude of the complex viscosity (η*) and the steady shear viscosity (η) must be superimposed at equal values of frequency and shear rate as presented by Eq. (7) (Cox & Merz, 1958). An important feature of this rule is the establishment of a correlation between large deformations, the steady shear flows, which are basically non-linear and the small and linear deformations, the small-amplitude oscillatory shears (SAOS) (Gunasekaran & Ak, 2000). A relationship between the steady shear and oscillatory data is valuable to correlate true material properties obtained from different tests (Gunasekaran & Ak, 2000; Steffe, 1996).

η*=η|_{ω=\dot{γ}} (7)

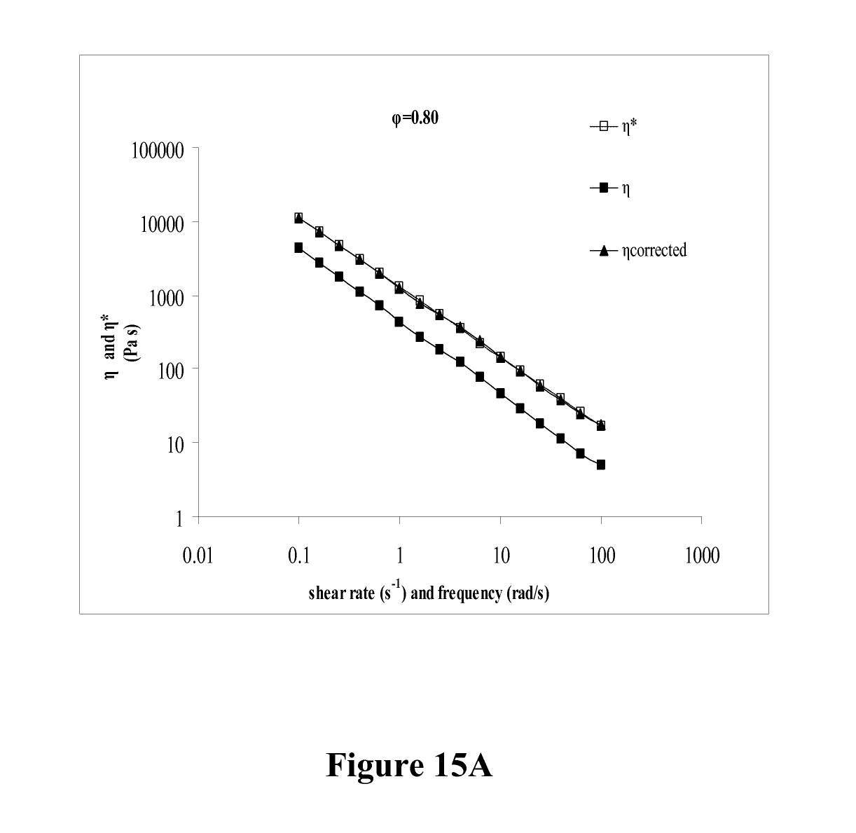

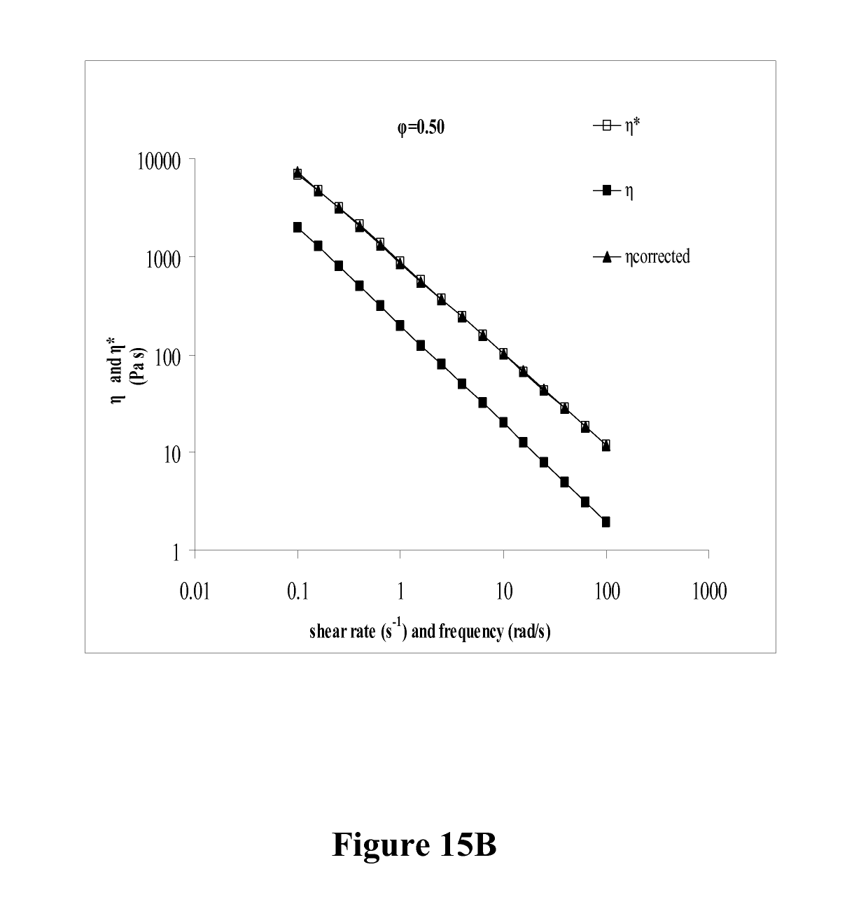

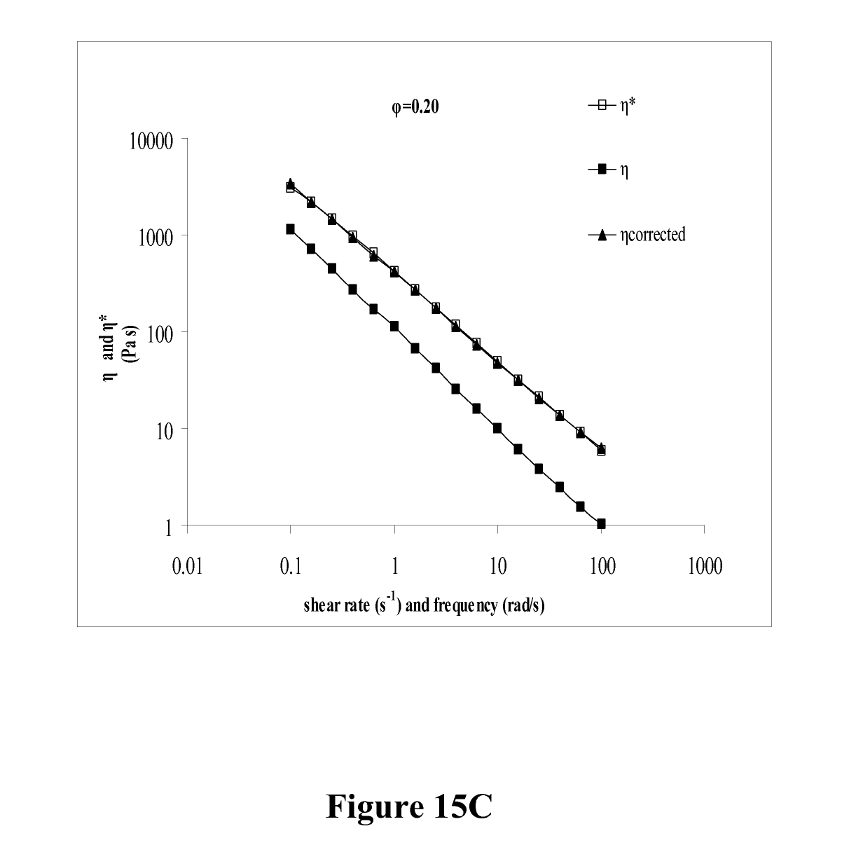

The power law parameters relating steady shear viscosity to shear rate and complex viscosity to frequency for tWPC-stabilized emulsions at various oil fractions are presented in Table 7. It shows that values of A were approximately 3 times less than values of B in all cases. The η* and η values of three emulsion samples (φ=0.20, 0.50, and 0.80) are presented in FIGS. 15A-15C as functions of frequency and shear rate. It illustrates that the Cox-Merz rule did not fit η* and η plotted at equivalent frequencies (0.1-100 rad/s) and shear rates (0.1-100 s⁻¹) for all tWPC-based emulsions. Parallel dependencies of η* on frequency and η on shear rate were obtained with the values of η* higher than the η values from continuous shear ramps (FIGS. 15A-15C). It means that samples did not hold the Cox-Merz rule. However, the result showed that the commercial WPC80-stabilized emulsion based on 80% (w/w) held the Cox-Merz rule over a shear rate/frequency range of 0.1-10 s⁻¹. Departures from this rule have been reported to occur in structured polymer systems such as in polymer liquid crystals or when aggregation takes place among polymer chains and highly-branched starch (Chamberlain & Rao, 2000; Lapasin & Pricl, 1995). Gunasekaran and Ak (2000) described that departures from Cox-Merz rule are attributed to structural decay due to the extensive strain applied. Though applied strain is low in SAOS, it is sufficient enough in steady shear to break down structured inter and intra-molecular associations of materials (Ahmed & Ramaswamy, 2006; Gunasekaran & Ak, 2000). According to some authors (Rao, Okechukwu, Da Silva, & Oliveira, 1997; Tárrega, Durán, & Costell, 2005), deviations from the Cox-Merz rule may imply strong inter and intra-molecular associations or a gel-like structure.

TABLE 7
Power law parameters for steady shear viscosity (η) versus shear rate (\dot{γ})
and complex viscosity (η*) versus frequency (ω) and modified
Cox-Merz rule parameters for emulsions stabilized with tWPC

| | | | |
|---|---|---|---|
| Oil | | Oscillatory | Mod. |
| mass | Steady shear | shear | Cox-Merz |
| fraction | (η = A\dot{γ}^{a}) | (η* = Bω^{b}) | (η* = Kη^{α}) |

| (φ) | A | a | B | b | K | α |
|---|---|---|---|---|---|---|
| 0.80 | 447.99 | 0.986 | 1290.60 | 0.946 | 3.76 | 0.957 |
| 0.70 | 307.33 | 0.955 | 985.89 | 0.939 | 3.55 | 0.982 |
| 0.60 | 205.11 | 0.935 | 877.28 | 0.928 | 4.47 | 0.992 |
| 0.50 | 198.37 | 1.004 | 861.33 | 0.929 | 6.45 | 0.925 |
| 0.40 | 103.54 | 1.004 | 455.14 | 0.911 | 6.76 | 0.907 |
| 0.30 | 105.06 | 1.008 | 482.13 | 0.918 | 6.96 | 0.910 |
| 0.20 | 107.01 | 1.024 | 404.36 | 0.915 | 6.20 | 0.894 |

Although, the Cox-Merz rule proved inadequate for studied emulsions, the modified version of this rule with the introduction of constant K (shift factor) and a values supplied a useful relationship (Eq. (8)). This is called generalized Cox-Merz rule (Gunasekaran & Ak, 2000). The suitability of the generalized Cox-Merz relation was studied by fitting both the variations of η* and η data with frequency/shear rate to the power law. Results revealed that the η* and η data were represented well by the generalized Cox-Merz relation (R²>0.999). The power law parameters (K and α) for emulsions based on tWPC are presented in Table 7.

η* =Kη^{α}|_{ω=\dot{γ}} (8)

The parameters, K and α, obtained from Eq. (8) can be implemented to compute a ‘corrected’ steady shear viscosity (η_{corrected}). The applicability of generalized Cox-Merz for studied emulsions is shown by the η_{corrected} values that match the complex viscosity over a shear rate/frequency range studied as presented in FIGS. 15A-15C. A generalized Cox-Merz relation was also observed to hold for several semi-solid food materials such as apple butter, mustard, cream cheese, margarine, sweet potato puree infant food, derivatized WP gels, and diary desserts (Ahmed & Ramaswamy, 2006; Bistany & Kokini, 1983; Resch, Daubert, & Foegeding, 2004; Tárrega et al., 2005; Yu & Gunasekaran, 2001).

### Thermal Stability Of Cold, Gel-Like Emulsions

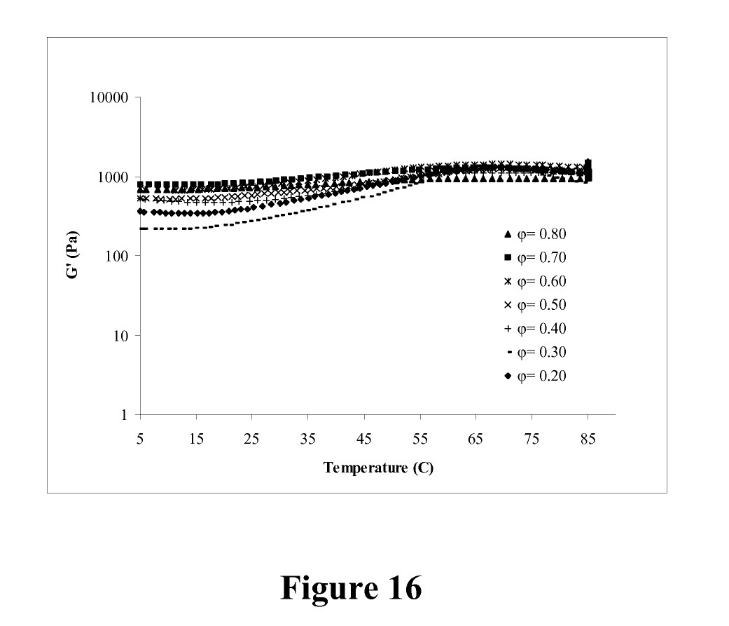

Results showed that emulsions stabilized by tWPC were far less sensitive to heat treatment than emulsions based on commercial WPC80. All emulsions based on tWPC displayed little variation in elastic modulus (G″) when the temperature was raised from 5° C. to 85° C. (FIG. 16). A slight variation in G′ of these emulsions after the temperature ramping could be attributed to the additional unfolding and denaturation of native WPs remaining in an aqueous phase. In previous work, our results showed that the SCFX process rendered WPC into a tWPC powder with cold-setting gel characteristics that was stable during heat treatment. A similar trend was expected for this study and all emulsions stabilized by tWPC were relatively stable over a wide range of temperature. On the other hand, most emulsions prepared with commercial WPC80 partially coagulated and exhibited a hard texture and phase separation after emulsions were heated to 85° C. This is obviously related to denaturation of the native WPs mainly in the commercial WPC80 sample.

### Conclusions

The experimentals provided in Examples 4-6 demonstrate that tWPC exhibited excellent emulsifying properties. It may be suggested that there are two possible stabilization mechanisms in the emulsion prepared with tWPC. First mechanism serves for emulsifying capability and the second mechanism serves for the stability effect, preventing creaming. These mechanisms, alone or synergistically, were responsible for the higher surface activity and emulsion stability of tWPC compared to that of commercial WPC80.

The homogeneous gel-like emulsions with various oil concentrations could be successfully produced at ambient temperature by incorporation of tWPC within an aqueous phase. Oil in water, gel-like emulsions based on tWPC were very stable and had finely dispersed fat droplets. Emulsions with higher apparent viscosity and elasticity were obtained by raising the oil concentration since oil droplets acted as active filler and strengthened the emulsions. The gel-like emulsions at all oil concentrations demonstrated markedly thermal stability indicated by little variation in rheological properties upon heating to 85° C. The results of this study confirmed our hypothesis that reactive SCFX rendered WPC into an ingredient with excellent gelling and emulsifying properties. This may open up a new avenue for utilization of tWPC in food emulsions, especially in food formulations containing heat-sensitive ingredients.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

### REFERENCES

Citation of a reference herein shall not be construed as an admission that such reference is prior art to the present invention. All references cited herein are hereby incorporated by reference in their entirety. Below is a listing of various references cited herein:
Aguilera, J. M., & Rojas, E. (1996). Rheological, thermal and microstructural properties of whey protein-cassava starch mixed gels. Journal of Food Science, 61, 963-966.
Ahmed, J., & Ramaswamy, H. S. (2006). Viscoelastic properties of sweet potato puree infant food. Journal of Food Engineering, 74, 376-382.
Akiyama, E., Kashimoto, A., Fukuda, K., Hotta, H., Suzuki, T., & Kitsuki, T. (2005). Thickening properties and emulsification mechanisms of new derivatives of polysaccharides in aqueous solution. Journal of Colloid and Interface Science, 282, 448-457.
Akiyama, E., Yamamoto, T., Yago, Y., Hotta, H., Ihara, T., & Kitsuki, T. (2007). Thickening properties and emulsification mechanisms of new derivatives of polysaccharide in aqueous solution 2. The effect of the substitution ratio of hydrophobic/hydrophilic moieties. Journal of Colloid and Interface Science, 311, 438-446.
Alavi, S. H., Gogoi, B. K., Khan, M., Bowman, B. J., & Rizvi, S. S. H. (1999). Structural properties of protein-stabilized starch-based supercritical fluid extrudates. Food Research International, 32, 107-118.
Alting, A. C. (2003). Cold gelation of globular proteins. PhD Thesis. Wageningen University, The Netherlands.
Amaya-Llano, S. L., Morales Hernández, N., Castaño Tostado, E., & Martínez-Bustos, F. (2007). Functional characteristics of extruded blends of whey protein concentrate and corn starch. Cereal Chemistry, 82, 195-201.
Barbut, S., & Foegeding, E. A. (1993). Ca²⁺-induced gelation of pre-heated whey protein isolate. Journal of Food Science, 58, 867-871.
Beaulieu, L., Savoie, L., Paquin, P., & Sabirade, M. (2002). Elaboration and characterization of whey protein beads by an emulsification/cold gelation process: application for the protection of retinol. Biomacromolecules, 3, 239-248.
Bécu, L., Manneville, S., & Colin, A. (2006). Yielding and Flow in Adhesive and Nonadhesive Concentrated Emulsions. Physical Review Letters, 96, 138302.
Bernal, V., & Jelen, P. (1984). Effect of calcium binding on thermal denaturation of bovine α-lacalbumin. Journal of Dairy Science, 67, 2452-2454.
Beveridge, T., & Timbers, G. E. (1985). Small amplitude oscillatory testing (SAOT). Instrumentation development and application to coagulation of egg albumin, whey protein concentrate and beef wiener emulsion. Journal of Texture Studies, 16, 333-348.
Bistany, K. L., & Kokini, J. L. (1983). Dynamic viscoelastic properties of foods in texture control. Journal of Rheology, 27, 605-620.
Bohlin, L. (1980). A theory of flow as a cooperative phenomenon. Journal of Colloid and Interface Science, 74, 423-434.
Boutin, C., Giroux, H. J., Paquin, P., & Britten, M. (2007). Characterization and acid-induced gelation of butter oil emulsions produced from heated whey protein dispersions. International Dairy Journal, 17, 696-703.
Bowland, E., & Foegeding, E. A. (1995). Effects of anions on thermally induced whey protein isolate gel. Food Hydrocolloids, 9, 47-56.
Boye, J. I., Kalab, M., Alli, I., & Ma, C. Y. (2000). Microstructural properties of heat-set whey protein gels: effect of pH. Lebensmittel-Wissenschaft und-Technologie, 33, 165-172.
Britten, M., Giroux, H. J., Jean, Y., & Rodrigue, N. (1994). Composite blends from heat-denatured and undenatured whey protein: emulsifying properties. International Dairy Journal, 4, 25-36.
Bryant, C. M., & McClements, D. J. (1998). Molecular basis of protein functionality with special consideration of cold-set gels derived from heat-denatured whey. Trends in food science and technology, 9, 143-151.
Campanella, O. H., Dorward, N. M., & Singh, H. (1995). A study of the rheological properties of concentrated food emulsions. Journal of Food Engineering, 25, 427-440.
Carvalho, C. W. P., Onwulata, C. I., & Tomasula, P. M. (2007). Rheological properties of starch and whey protein isolate gels. Food Science and Technology International, 13, 207-216.
Chamberlain, E. K., & Rao, M. A. (2000) Rheological properties of acid converted waxy maize starches. Carbohydrate Polymers, 40, 251-260.
Chen, J., & Dickinson, E. (1998). Viscoelastic properties of heat-set whey protein emulsion gels. Journal of Texture Studies, 29, 285-304.
Chen, J., & Dickinson, E. (1999). Effect of monoglycerides and diglycerol-esters on viscoelasticity of heat-set whey protein emulsion gels. International Journal of Food Science and Technology, 34, 493-501.
Chen, K. H. (2005). Behaviors of controlled starch gelatization in supercritical fluid extrusion. PhD Thesis, Cornell University, USA.
Clark, A. H. (1998). Gelation of globular proteins. In S. E. Hill, D. A. Leward, & J. R. Mitchell (Eds.), Functional properties of food macromolecules (pp. 77-138). Maryland, USA: Aspen Publishers, Inc.
Clark, A. M., & Ross-Murphy, S. B. (1987). Structural and mechanical properties of biopolymer gels. Advances in Polymer Science, 83, 57-192.
Cox, W. P., & Merz, E. H. (1958). Correlation of dynamic and steady flow viscosities. Journal of Polymer Science, 28, 619-622.
Curran, S. J., Hayes, R. E., Afacan, A., Williams, M. C., & Tanguy, P. A. (2002). Properties of carbopol solutions as models for yield-stress fluids. Journal of Food Science, 67, 176-180.
Dagorn-Scaviner, C., Guéguen, J., & Lefebre, J. (1987). Emulsifying properties of pea globulins as related to their adsorption behaviors. Journal of Food Science, 52, 335-341.
Dahl, S. R., & Villota, R. (1991). Twin-screw extrusion texturization of acid and alkali denatured soy proteins. Journal of Food Science, 56, 1002-1007.
Dalgleish, D. G. (1997). Adsorption of protein and the stability of emulsions. Trends in Food Science & Technology, 8, 1-6.
Dalgleish, D. G. (2006). Food emulsions-their structures and structure-forming properties. Food Hydrocolloids, 20, 415-422.
Dalgleish, D. G., & Singh, A. M. (1998). The emulsifying properties of hydrolysates of whey proteins. Journal of Dairy Science, 81, 918-924.
Daubert, C. R., Resch, J. J., & Foegeding, E. A. (2004). A comparison of drying operations on the rheological properties of whey protein thickening ingredients. International Journal of Food Science and Technology, 39, 1023-1031.
Demetriades, K., Coupland, J. N., and McClements, D. J. (1997). Physical properties of whey protein stabilized emulsions as related to pH and NaCl. Journal of Food Science, 62, 342-347.
Demetriades, K., & McClements, D. J. (1998). Influences of pH and heating on physicochemical properties of whey protein-stabilized emulsions containing nonionic surfactant. Journal of Agricultural and Food Chemistry, 46, 3936-3942.
Depree, J. A., & Savage, G. P. (2001). Physical and flavour stability of mayonnaise. Trends in Food Science & Technology, 12, 157-163.
de Wit, J. N., Klarenbeek, G. (1984). Effects of various heat treatments on structure and solubility of whey proteins. Journal of Dairy Science, 67, 2701-2710.
Dickinson, E. (1995). Emulsion stabilization by polysaccharides and protein-polysaccharide complexes. In A. M. Stephen, Food polysaccharides and their applications (pp. 501-515). New York: Marcel Dekker Inc.
Dickinson, E. (1997). Properties of emulsions stabilized with milk proteins: overview of some recent developments. Journal of Dairy Science, 80, 2607-2619.
Dickinson, E., & Hong, S. T. (1995). Influence of water-soluble non-ionic emulsifier on the rheology of heat-set protein stabilized emulsion gels. Journal of Agricultural and Food Chemistry, 43, 2560-2566.
Dickinson, E., & Hong, S. T. (1997). Influence of anionic surfactant on the rheology of heat-set β-lactoglobulin-stabilized emulsion gels. Colloids and Surfaces A, 127, 1-10.
Dimitrova, T. D., & Leal-Calderon, F. (2004). Rheological properties of highly concentrated protein-stabilized emulsions. Advances in Colloid and Interface Science, 108-109, 49-61.
El-Garawany, G. A., & Abd El Salam, M. H. (2005). Preparation and rheological properties of a dairy dessert based on whey protein/potato starch. Food Chemistry, 91, 261-267.
Elofsson, C., Dejmek, P., Paulsson, M., & Burling, H. (1997). Characterization of a cold-gelling whey protein concentrate. International Dairy Journal, 7, 601-608.
Euston, S. R., & Hirst, R. L. (1999). Comparison of the concentration-dependent emulsifying properties of protein products containing aggregates and non-aggregated milk protein. International Dairy Journal, 9, 693-701.
Eynard, L., Iametti, S., Relkin, P., & Bonomi, F. (1992). Surface hydrophobicity changes and heat-induced modifications of α-Lacalbumin. Journal of Agricultural and Food Chemistry, 40, 1731-1736.
Fachin, L., & Viotto, W. H. (2005). Effect of pH and heat treatment of cheese whey on solubility and emulsifying properties of whey protein concentrate produced by ultrafiltration. International Dairy Journal, 15, 325-332.
Firebaugh, J. D. (2004). Characterization and application of a derivatized whey ingredient. MS thesis, North Carolina State University, USA.
Firebaugh, J. D., & Daubert, C. R. (2005). Emulsifying and foaming properties of derivatized whey protein ingredient. International Journal of Food properties, 8, 243-253.
Fleming, S. E., Sosulski, F. W., Kilara, A., & Humbert, E. S. (1974). Viscosity and water absorption characteristics of slurries of sunflower and soybean flours, concentrates, and isolates. Journal of Food Science, 39, 188-192.
Gerhards, Ch., & Schubert, H. (1993). Viscoelastic and shear thinning behavior of processed mustard. Rheology, 12, 256-263.
Graham, D. E., & Phillips, M. C. (1976). The conformation of proteins at interfaces and their role in stabilizing emulsions. In A. L. Smith (Ed.), Theory and Practice of Emulsion Technology; (pp. 75-93). London: Academic Press.
Graham, D. E., & Phillips, M. C. (1980). Protein at liquid interfaces. Part 5. Journal of Colloid and Interface Science, 76, 240-252.
Guilmineau, F., & Kulozik, U. (2007). Influence of a thermal treatment on the functionality of hen's egg yolk in mayonnaise. Journal of Food Engineering, 78, 648-654.
Gunasekaran, S., & Ak, M. M. (2000). Dynamic oscillatory shear testing of foods-selected applications. Trends in Food Science & Technology, 11, 115-127.
Hale, A. B., Carpenter, C. E., Walsh, M. K. (2000). Instrumental and consumer evaluation of beef patties extended with extrusion-texturized whey proteins. Journal of Food Science, 67, 1267-1270.
Harding, S. E. (1998). Dilute solution viscometry of food biopolymers. In S. E. Hill, D. A. Ledward, & J. R. Mitchell (Eds.), Functional properties of food macromolecules (2nd ed) (pp. 1-43). Gaithersburg, Md., USA: Aspen Publisher, Inc.
Hemar, Y., & Horne, D. S. (2000). Dynamic rheological properties of highly concentrated protein-stabilized emulsions. Langmuir, 16, 3050-3057.
Hudson, H. M., & Daubert, C. R. (2002). Functionality comparison between derivatized whey proteins and a pregelatinized starch. Journal of Texture Studies, 33, 297-314.
Hudson, H. M., Daubert, C. R., & Foegeding, E. A. (2000). Rheological and physical characterization of derivatized whey protein powders. Journal of Agricultural and Food Chemistry, 48, 3112-3119.
Hunt, J. A., & Dalgleish, D. G. (1994). Effect of pH on the stability and surface composition of emulsions made with whey protein isolate. Journal of Agricultural and Food Chemistry, 42, 2131-2135.
Ikeda, S., & Morris, V. J. (2002). Fine-stranded and particulate aggregates of heat-denatured whey proteins visualized by atomic force microscopy. Biomacromolecules, 3, 382-389.
Jelen, P., & Buchheim, W. (1984). Stability of whey protein upon heating in acidic conditions. Milchwissenschaft, 39, 215-218.
Jost, R., Baechler, R., & Masson, G. (1986). Heat gelation of oil-in-water emulsions stabilized by whey protein. Journal of Food Science, 51, 440-444, 449.
Jost, R., Dannenberg, F., & Rosset, J. (1989). Heat-set based on oil/water emulsions: an application of whey protein functionality. Food Microstructure, 8, 23-28.
Ju, Z. Y., & Kilara, A. (1998a). Gelation of pH-aggregated whey protein isolate induced by heat, protease, calcium salt, and acidulant. Journal of Agricultural and Food Chemistry, 46, 1830-1835.
Ju, Z. Y., & Kilara, A. (1998b). Effects of preheating on properties of aggregates and of cold-set gels of whey protein isolate. Journal of Agricultural and Food Chemistry, 46, 3604-3608.
Kato, A., & Nakai, S. (1980). Hydrophobicity determined by fluorescence probe method and its correlation with surface properties of proteins. Biochemica et Biophysica Acta, 624, 13-20.
Kella, N. K. D., Yang, S. T., & Kinsella, J. E. (1989). Effect of disulfide bond cleavage on structural and interfacial properties of whey proteins. Journal of Agricultural and Food Chemistry, 37, 1203-1210.
Kester, J. J., & Richardson, T. (1984). Modification of whey proteins to improve functionality. Journal of Dairy Science, 67, 2757-2774.
Kim, C. H., & Maga, J. A. (1987). Properties of extruded whey protein concentrate and cereal flour blends. Labensmittel-Wissenchaftund-Technologie, 20, 311-318.
Kinsella, J. E. (1979). Functional properties of soy proteins. Journal of the American Oil Chemists' Society, 56, 242-258.
Kinsella, J. E., & Whitehead, D. M. (1989). Proteins in whey: chemical, physical and functional properties. Advances in Food and Nutrition Research, 33, 344-430.
Klemaszewski, J. L., Das, K. P., Kang, Y. J., & Kinsella, J. E. (1990). Effects of controlled sulfitolysis of bovine serum albumin on droplet size and surface area of emulsions. Journal of Agricultural and Food Chemistry, 38, 647-650.
Klemaszewski, J. L., & Kinsella, J. E. (1991). Sulfitolysis of whey proteins: effects on emulsion properties. Journal of Agricultural and Food Chemistry, 39, 1033-1036.
Kuehler, C. A., & Stine, C. M. (1974). Effect of enzymatic hydrolysis on some functional properties of whey protein. Journal of Food Science, 39, 379-382.
Lang, E. R. B., & Rha, C. (1981). Determination of the yield stress of hydrocolloid dispersions. Journal of Texture Studies, 12, 47-62.
Lapasin, R., & Pricl, S. (1995). Rheology of industrial polysaccharides, Theory and applications, Blackie Academic & Professional, Chapman & Hall, London.
Li-Chan, E., Nakai, S., & Wood, D. F., (1984). Hydrophobicity and solubility of meat proteins and their relationship to emulsifying properties. Journal of Food Science, 49, 345-350.
Liu, H. L., Hsieh, W. C., & Liu, H. S. (2004). Molecular dynamics simulations to determine the effect of supercritical carbon dioxide on the structural integrity of hen egg white lysozyme. Biotecnology Progress, 20, 930-938.
Liu, H., Xu, X. M., & Guo, Sh. D. (2007). Rheological, texture and sensory properties of low-fat mayonnaise with different fat mimetics. Lebensmittel-Wissenschaft und-Technologie, 40, 946-954.
Lucassen-Reynders, E. H. (1993). Interfacial viscoelasticity in emulsions and foams. Food Structure, 12, 1-12.
Ma, L., & Barbosa-Cánovas, G. V. (1995). Rheological characterization of mayonnaise. Part II: flow and viscoelastic properties at different oil and xanthan gum concentrations. Journal of Food Engineering, 25, 409-425.
Mangino, M. E. (1984). Physicochemical aspects of whey protein functionality. Journal of Dairy Science, 67, 2711-2722.
Mangino, M. E. (1992). Gelation of whey protein concentrates. Food Technology, 46, 114-117.
Manoi, K., & Rizvi, S. S. H. (2008). Rheological characterizations of texturized whey protein concentrate-based powders produced by reactive supercritical fluid extrusion. Food Research International, 41, 786-796.
Martinez-Serna, M. D. & Villota, R. (1992). Reactivity, functionality, and extrusion performance of native and chemically modified whey proteins. In J. L. Kokini, C. T. Ho, & M. V. Karwe (Eds.), Food extrusion science and technology (pp. 387-414). New York, N.Y., USA: Marcel Dekker, Inc.
Matsudomi, N., Sasaki, T., Kato, A., & Kobayashi, K. (1985). Conformational changes and functional properties of acid-modified soy protein. Agricultural and Biological Chemistry, 49, 1251-1256.
Matsumura, Y., Kang, I., Sakamoto, -J., Motoki, H., & Mori, T. (1993). Filler effects of oil droplets on the viscoelasticity of emulsion gels. Food Hydrocolloid, 7, 227-240.
McClements, D. J. (1999a). Emulsion Rheology. In D. J. McClements (Ed.), Food Emulsions-Principles, practice and techniques (pp. 235-266). Washington, D.C.: CRC Press.
McClements, D. J. (1999b). Food Emulsions: principles, practice and techniques. New York: CRC Press.
McClements, D. J. (2000). Comments on viscosity enhancement and depletion flocculation by polysaccharides. Food Hydrocolloids, 14, 173-177.
Michael, H. T., & Onwulata, C. I. (2006). Rheological properties of extruded milk powders. International Journal of Food Properties, 9, 835-844.
Mitidieri, F. E., & Wagner, J. R. (2002). Coalescence of o/w emulsions stabilized by whey and isolate soybean proteins. Influence of thermal denaturation, salt addition and competitive interfacial adsorption. Food Research International, 35, 547-557.
Monahan, F. J., German, J. B., & Kinsella, J. E. (1995). Effects of pH and temperature on protein unfolding and thiol/disulfide interchange reactions during heat-induced gelation of whey proteins. Journal of Agricultural and Food Chemistry, 43, 46-52.
Monti, J. C., & Jost, R. (1978). Enzymatic solubilization of heat-denatured cheese whey protein. Journal of Dairy Science, 61, 1233-1237.
Mulvaney, S. J., & Rizvi, S. S. H. (1993). Extrusion processing with supercritical fluids. Food Technology, 47, 74-82.
Mulvihill, D. M., & Kinsella, J. E. (1987). Gelation characteristics of whey proteins and β-lactoglobulin. Food Technology, 41, 102-111.
Nakai, S., & Li-Chan, E. (1984). Structure modification and functionality of whey proteins: quantitative structure-activity relationship approach. Journal of Dairy Science, 68, 2763-2772.
Onwulata, C. I., Isobe, S., Tomasula, P. M., & Cooke, P. H. (2006). Properties of whey protein isolates extruded under acidic and alkaline conditions. Journal of Dairy Science, 89, 71-81.
Onwulata, C. I., Konstance, R. P., Cooke, P. H., & Farrell, H. M. (2003). Functionality of extrusion-texturized whey proteins. Journal of Dairy Science, 86, 3775-3782.
Palazolo, G. G., Sorgentini, D. A., & Wagner, J. R. (2004). Emulsifying properties and surface behavior of native and denatured whey soy proteins in comparison with other proteins. Creaming stability of oil-in-water emulsions. Journal of the American Oil Chemists' Society, 81, 625-632.
Patel, M. T., & Kilara, A. (1990). Studies on whey protein concentrates. 2. Foaming and emulsifying properties and their relationship with physicochemical properties. Journal of Dairy Science, 73, 2731-2740.
Paulsson, M., Hegg, P.-O., & Castberg, H. B. (1986). Heat-induced gelation of individual whey proteins. A dynamic rheological study. Journal of Food Science, 51, 87-90.
Pearce, K. N., & Kinsella, J. E. (1978). Emulsifying properties of proteins: evaluation of a turbidity technique. Journal of Agricultural and Food Chemistry, 26, 716-723.
Peressini, D., & Sensidoni, A. (2000). Rheology of wheat doughs for fresh pasta production: influence of semolina-flour blends and salt content. Journal of Texture Studies, 31, 163-182.
Peressini, D., Sensidoni, A., & de Cindio, B. (1998). Rheological characterization of traditional and light mayonnaises. Journal of Food Engineering, 35, 409-417.
Puppo, M. C., Sorgentini, D. A., & Anon, M. C. (2003). Rheological properties of emulsions containing modified soy protein isolates. Journal of the American Oil Chemists' Society, 80, 605-611.
Queguiner, C., Dumay, E., Salou-Cavalier, C., & Cheftel, J. C. (1992). Microcoagulation of a whey protein isolate by extrusion cooking at acid pH. Journal of Food Science, 57, 610-616.
Rattray, W., & Jelen, P. (1995). Viscous behavior of whey protein concentrate dispersions. International Dairy Journal, 5, 673-684.
Rao, M. A., Okechukwu, P. E., Da Silva, P. M. S., & Oliveira, J. C. (1997). Rheological behavior of heated starch dispersions in excess water: role of starch granule. Carbohydrate Polymers, 33, 273-283.
Raymundo, A., Franco, J. M., Empis, J., & Sousa, I. (2002). Optimization of the composition of low-fat oil-in-water emulsions stabilized by white lupin protein. Journal of the American Oil Chemists' Society, 79, 783-790.
Rector, D. J. (1992). Polymer properties of whey proteins, gelation, viscous and glassy behavior. MS Thesis, Cornell University, USA.
Resch, J. J., Daubert, C. R., & Foegeding, E. A. (2004). A comparison of drying operations on the rheological properties of whey protein thickening ingredients. International Journal of Food Science and Technology, 39, 1023-1031.
Resch, J. J. (2004). Processing parameter effects on the functional properties of derivatized whey protein ingredients. PhD Thesis, North Carolina State University, USA.
Rodd, A. B., Davis, C. R., Dunstan, D. E., Forrest, B. A., & Boger, D. V. (2000). Rheological characterization of ‘weak gel’ carrageenan stabilized milks. Food Hydrocolloids, 14, 445-454.
Roland, I., Piel, G., Delattre, L., & Evrard, B. (2003). Systematic characterization of oil-in-water emulsions for formulation design. International Journal of Pharmaceutics, 263, 85-94.
Rosa, P., Sala, G., Van Vliet, T., & Van De Velde, F. (2006). Cold gelation of whey protein emulsions. Journal of Texture Studies, 37, 516-537.
Sato, K., Nakamura, M., Nishiya, T., Kawanari, M., & Nakajima, I. (1995). Preparation of a gel of partially heat-denatured whey proteins by proteolytic digestion. Milchwissenschaft, 50, 389-392.
Schmidt, R. D., Packard, V. S., & Morris, H. A. (1984). Effect of processing on whey protein functionality. Journal of Dairy Science, 67, 2723-2733.
Schmidt, R. H., Packard, V. S., & Morris, H. A. (1983). Effect of processing on whey protein functionality. Journal of Dairy Science, 67, 2723-2733.
Shim, J., & Mulvaney, S. J. (2001). Effect of heating temperature, pH, concentration and starch/whey protein ratio on the viscoelastic properties of corn starch/whey protein mixed gels. Journal of the Science of Food and Agriculture, 81, 706-717.
Singer, N. S., Yamamoto, S., & Latella, J. (1988). Protein product base. U.S. Pat. No. 4,734,287.
Sok Line, V. L., Remondetto, G. E., & Suburade, M. (2005). Cold gelation of β-lactoglobulin oil-in-water emulsions. Food Hydrocolloids, 19, 269-278.
Steffe, J. F. (1996). Rheological methods in food processing (2nd ed.). East Lansing, Mich.: Freeman Press.
Striolo, A., Favaro, A., Elvassore, N., Bertucco, A., & Di Noto, V. (2003). Evidence of conformational changes for protein films exposed to high-pressure CO₂ by FT-IR spectroscopy. Journal of Supercritical Fluid, 27, 283-295.
Sun, D., Sun, W., Wei, Y., Liu, S., & Zhang, S. (2007). Oil-in-water emulsions stabilized by hydrophobically modified hydroxyethyl cellulose. Journal of Colloid and Interface Science, 311, 228-236.
Tang, Q., McCarthy, O., & Munro, P. (1995). Oscillatory study of the effect of pH and salts on gel development in heated whey protein concentrate solutions. Journal of Diary Research, 62, 469-477.
Tárrega, A., Durán, L., & Costell, E. (2005). Rheological characterization of semisolid dairy desserts. Effect of temperature. Food Hydrocolloids, 19, 133-139.
Taulier, N., & Chalikian, T. V. (2001). Characterization of pH-induced transitions of β-lactoglobulin: ultrasonic, densimetric, and spectroscopic studies. Journal of Molecular Biology, 314, 873-889.
Thomsen, B. (1994). Whey protein texturizer. European Food and Drink Review, Spring, 46-47.
Turgeon, S. L., Sanchez, C., Gauthier, S. F., & Paquin, P. (1996). Stability and rheological properties of salad dressing containing peptidic fractions of whey proteins. International Dairy Journal, 6, 645-658.
Voutsinas, L. P., & Nakai, S. (1983). A simple turbidimetric method for determining the fat binding capacity of proteins. Journal of Agricultural and Food Chemistry, 31, 58-63.
Vreeker, R., Hoekstra, L. L., den Boer, D. C., & Agterof, W. G. M. (1992). Fractal aggregation of whey proteins. Food Hydrocolloids, 5, 423-435.
Wagner, J. R., & Guéguen, J. (1999). Surface functional properties of native, acid-treated and reduced soy glycinin. 2. emulsifying properties. Journal of Agricultural and Food Chemistry, 47, 2181-2187.
Wagner, J. R., Sorgentini, D. A., & Añón, M. C. (1996). Thermal and electrophoretic behavior, hydrophobicity, and some functional properties of acid-treated soy isolates. Journal of Agricultural and Food Chemistry, 44, 1881-1889.
Walsh, M. K., & Carpenter C. E. (2003). Textured whey protein product and method. U.S. Pat. No. 6,607,777.
Watanabe, K., & Klostermeyer, H. (1976). Heat-induced changes in sulfhydryl and disulfide levels of β-lactoglobulin A and formation of polymers. Journal of Dairy Research, 43, 411-418.
Yamauchi, K., Shimizu, M., & Kamiya, T. (1980). Emulsifying properties of whey proteins. Journal of Food Science, 45, 1237-1242.
Yost, R. A., & Kinsella, J. E. (1992). Microstructure of whey protein isolate gels containing emulsified butterfat droplets. Journal of Food Science, 57, 892-897.
Yu, Ch., & Gunasekaran, S. (2001). Correlation of dynamic and steady flow viscosities of food materials. Applied Rheology, 11, 134-140.
Zhong, Q., & Jin, M. (2008). Enhanced functionalities of whey proteins treated with supercritical carbon dioxide. Journal of Dairy Science, 91, 490-499.
Ziegler, G. R., & Foegeding, E. A. (1990). The gelation of proteins. Advances in Food and Nutrition Research, 34, 203-298.

What is claimed is:

1. A texturized whey protein composition comprising:
a supercritical fluid extrusion product derived from whey protein.

2. The composition according to claim 1, wherein said texturized whey protein composition is effective as a food preparation agent selected from the group consisting of a thickening agent, a gelling agent, an emulsifier, a stabilizer, and a water binder.

3. The composition according to claim 1, wherein said supercritical fluid extrusion product comprises about 70 or more weight percent of a whey protein concentrate.

4. The composition according to claim 1, wherein said supercritical fluid extrusion product comprises a whey protein concentrate and an edible polysaccharide component.

5. The composition according to claim 4, wherein said edible polysaccharide component is a starch selected from the group consisting of corn, potato, rice, tapioca, bran, and soy starches, modified variants thereof, and mixtures thereof.

6. The composition according to claim 4, wherein said edible polysaccharide component is present in an amount of about 15 or less weight percent.

7. The composition according to claim 4, wherein said whey protein concentrate is present in an amount of between about 70 and about 100 weight percent and said edible polysaccharide component is present in an amount of between about 0 and about 15 weight percent.

8. The composition according to claim 1, wherein a reference sample having between about 10 and about 20 weight percent of said texturized whey protein composition has an apparent viscosity of between about 0.07 and about 2.06 pascal-seconds (Pa·s) measured at a shear rate of about 25 s⁻¹ and at ambient temperature.

9. The composition according to claim 1, wherein a reference sample having an oil concentration of about 20 percent weight-in-weight (w/w) and having about 3 percent weight-in-weight (w/w) of said texturized whey protein composition in an aqueous phase has an emulsifying activity index (EAI) of at least about 22 m²/g and an emulsion stability index (ESI) of at least about 30 or more hours measured at ambient temperature.

10. A food product comprising:
a texturized whey protein composition according to claim 1.

11. The food product according to claim 10, wherein said food product is selected from the group consisting of a protein-enriched food product, a nutrition bar, a dietary supplement, a reduced fat formulated food product, and the like.

12. A food preparation agent comprising:
a texturized whey protein composition according to claim 1, wherein said food preparation agent is selected from the group consisting of a thickening agent, a gelling agent, a stabilizer, and a water binder.

13. An emulsifier for use in food preparations, said emulsifier comprising:
a texturized whey protein composition according to claim 1.

14. An oil-in-water emulsion comprising:
an aqueous phase containing a food emulsifier according to claim 13; and
an oil phase dispersed in the aqueous phase.

15. The emulsion according to claim 14, wherein the texturized whey protein composition is present in an amount of between about 4 and about 16 weight percent.

16. A food product comprising:
an oil-in-water emulsion according to claim 14.

17. The food product according to claim 16, wherein the food product is selected from the group consisting of mayonnaise, margarine, a butter spread, a butter-like spread, a salad dressing, a texturizing cream product, a reduced fat formulated food product, a whipping cream, and the like.

18. The food product according to claim 16, wherein the food product comprises food formulations containing heat-sensitive ingredients.

19. A method of preparing an edible cold-setting gel-like emulsion, said method comprising:
providing an aqueous phase containing a texturized whey protein composition according to claim 1; and
dispersing an oil phase in the aqueous phase under conditions effective to yield an edible cold-setting gel-like emulsion.

20. The method according to claim 19, wherein the aqueous phase comprises between about 4 and about 16 weight-in-weight (w/w) percent of the texturized whey protein composition.

21. The method according to claim 19, wherein said oil phase comprises an oil selected from the group consisting of an animal-derived oil and a plant-derived oil.

22. A process for preparing a texturized whey protein composition, said process comprising:
providing a whey protein mixture comprising a whey protein concentrate; and
subjecting the whey protein mixture to a supercritical fluid extrusion (SCFX) process under conditions effective to yield a texturized whey protein composition having enhanced functional properties compared to a non-texturized whey protein composition.

23. The process according to claim 22, wherein said enhanced functional properties of the texturized whey protein composition are selected from the group consisting of enhanced viscosifying properties, enhanced emulsifying properties, and enhanced gelling properties.

24. The process according to claim 22, wherein said SCFX process is carried out at a temperature of between about 15° C. and about 100° C. and at a pH of about 8.4 or lower.

25. The process according to claim 22, wherein said SCFX process is carried out at ambient temperature.

26. The process according to claim 22, wherein said SCFX process is carried out at a pH of between about 2.80 and about 3.00.

27. The process according to claim 22, wherein said SCFX process comprises adjusting the pH of the whey protein mixture to a value of about 3.5 or lower, wherein adjusting the pH is performed by adding an acid, introducing carbon dioxide under high pressure, or a combination thereof.

28. The process according to claim 22, wherein said SCFX process is carried out at a pH of about 3.5 or lower, at a temperature of between about 85° C. and about 95° C., and at a pressure of between about 10 and 15 megapascal (MPa).

29. The process according to claim 22, wherein said SCFX process comprises introducing supercritical fluid carbon dioxide (SC—CO₂) under conditions effective to aid in the production of the texturized whey protein composition.

30. The process according to claim 22, wherein said whey protein mixture comprises about 70 or more weight percent of the whey protein concentrate.

31. The process according to claim 22, wherein a reference sample having between about 10 and about 20 weight percent of said texturized whey protein composition has an apparent viscosity of between about 0.07 and about 2.06 pascal-seconds (Pa·s) measured at a shear rate of about 25 s⁻¹ and at ambient temperature.

32. The process according to claim 22, wherein a reference sample having an oil concentration of about 20 percent weight-in-weight (w/w) and having about 3 percent weight-in-weight (w/w) of said texturized whey protein composition in an aqueous phase has an emulsifying activity index (EAI) of at least about 22 m²/g and an emulsion stability index (ESI) of at least about 30 or more hours measured at ambient temperature.

33. The process according to claim 22, wherein said whey protein mixture further comprises:
an edible polysaccharide component.

34. The process according to claim 33, wherein the edible polysaccharide component is a starch selected from the group consisting of corn, potato, rice, tapioca, bran, and soy starches, modified variants thereof, and mixtures thereof.

35. The process according to claim 33, wherein said edible polysaccharide component is present in an amount of about 15 or less weight percent.

36. The process according to claim 33, wherein said whey protein mixture comprises between about 70 and about 100 weight percent of the whey protein concentrate and between about 0 and about 15 weight percent of the edible polysaccharide component.

37. The process according to claim 22, wherein said whey protein mixture further comprises:
at least one inorganic element-containing compound in an amount sufficient to prevent excessive aggregation and to promote a stronger structure of protein gels, wherein said inorganic element is selected from the group consisting of calcium, sodium, potassium, magnesium, zinc, manganese, and the like.

38. The process according to claim 37, wherein said at least one inorganic element-containing compound is selected from the group consisting of calcium carbonate, calcium chloride, calcium gluconate, calcium lactate, sodium chloride, sodium citrate, sodium acetate, sodium lactate, and mixtures thereof.

39. The process according to claim 22 further comprising:
drying the texturized whey protein composition.

40. The process according to claim 22 further comprising:
grinding the texturized whey protein composition into a powder.

41. A texturized whey protein composition prepared by the process according to claim 22.
